(19) United States
(12) Patent Application Publication
(10) Pub. No.: US 2023/0234969 A1
(43) Pub. Date: Jul. 27, 2023

(54) N-LINKED MACROCYCLIC 7-(PYRAZOL-5-YL)-INDOLE DERIVATIVES AS INHIBIORS OF MCL-1

(71) Applicant: Janssen Pharmaceutica NV, Beerse (BE)

(72) Inventors: Frederik Jan Rita ROMBOUTS, Wilrijk (BE); Benoit Christian Albert Ghislain DE BOECK, Genval (BE); Aldo PESCHIULLI, Antwerp (BE); Adriana-Ingrid VELTER, Antwerpen (BE)

(21) Appl. No.: 18/001,352

(22) PCT Filed: Jun. 18, 2021
(86) PCT No.: PCT/EP2021/066653

§ 371 Date: Dec. 9, 2022

(30) Foreign Application Priority Data
Jun. 19, 2020 (EP) 20181138.7
Oct. 26, 2020 (EP) 20203938.4

(51) Int. Cl. C07D 515/22

(52) U.S. Cl. CPC C07D 515/22

(57) ABSTRACT

The present invention relates to pharmaceutical agents useful for therapy and/or prophylaxis in a subject, pharmaceutical composition comprising such compounds, and their use as MCL-1 inhibitors, useful for treating diseases such as cancer.

### FIELD OF THE INVENTION

The present invention relates to pharmaceutical agents useful for therapy and/or prophylaxis in a subject, pharmaceutical composition comprising such compounds, and their use as MCL-1 inhibitors, useful for treating or preventing diseases such as cancer.

### BACKGROUND OF THE INVENTION

Cellular apoptosis or programmed cell death is critical to the development and homeostasis of many organs including the hematopoietic system. Apoptosis can be initiated via the extrinsic pathway, which is mediated by death receptors, or by the intrinsic pathway using the B cell lymphoma (BCL-2) family of proteins. Myeloid cell leukemia-1 (MCL-1) is a member of the BCL-2 family of cell survival regulators and is a critical mediator of the intrinsic apoptosis pathway. MCL-1 is one of five principal anti-apoptotic BCL-2 proteins (MCL-1, BCL-2, BCL-XL, BCL-w, and BFL1/A1) responsible for maintaining cell survival. MCL-1 continuously and directly represses the activity of the pro-apoptotic BCL-2 family proteins Bak and Bax and indirectly blocks apoptosis by sequestering BH3 only apoptotic sensitizer proteins such as Bim and Noxa. The activation of Bak/Bax following various types of cellular stress leads to aggregation on the mitochondrial outer membrane and this aggregation facilitates pore formation, loss of mitochondrial outer membrane potential, and subsequent release of cytochrome C into the cytosol. Cytosolic cytochrome C binds Apaf-1 and initiates recruitment of procaspase 9 to form apoptosome structures (Cheng et al. eLife 2016; 5: e17755). The assembly of apoptosomes activates the executioner cysteine proteases 3/7 and these effector caspases then cleave a variety of cytoplasmic and nuclear proteins to induce cell death (Julian et al. Cell Death and Differentiation 2017; 24, 1380-1389).

Avoiding apoptosis is an established hallmark of cancer development and facilitates the survival of tumor cells that would otherwise be eliminated due to oncogenic stresses, growth factor deprivation, or DNA damage (Hanahan and Weinberg. Cell 2011; 1-44). Thus, unsurprisingly, MCL-1 is highly upregulated in many solid and hematologic cancers relative to normal non-transformed tissue counterparts. The overexpression of MCL-1 has been implicated in the pathogenesis of several cancers where it correlated with poor outcome, relapse, and aggressive disease. Additionally, overexpression of MCL-1 has been implicated in the pathogenesis of the following cancers: prostate, lung, pancreatic, breast, ovarian, cervical, melanoma, B-cell chronic lymphocytic leukemia (CLL), acute myeloid leukemia (AML), and acute lymphoblastic leukemia (ALL). The human MCL-1 genetic locus (1q21) is frequently amplified in tumors and quantitatively increases total MCL-1 protein levels (Beroukhim et al. Nature 2010; 463 (7283) 899-905). MCL-1 also mediates resistance to conventional cancer therapeutics and is transcriptionally upregulated in response to inhibition of BCL-2 function (Yecies et al. Blood 2010; 115 (16)3304-3313).

A small molecule BH3 inhibitor of BCL-2 has demonstrated clinical efficacy in patients with chronic lymphocytic leukemia and is FDA approved for patients with CLL or AML (Roberts et al. NEJM 2016; 374:311-322). The clinical success of BCL-2 antagonism led to the development of several MCL-1 BH3 mimetics that show efficacy in preclinical models of both hematologic malignancies and solid tumors (Kotschy et al. Nature 2016; 538 477-486, Merino et al. Sci. Transl. Med; 2017 (9)).

MCL-1 regulates several cellular processes in addition to its canonical role in mediating cell survival including mitochondrial integrity and non-homologous end joining following DNA damage (Chen et al. JCI 2018; 128(1):500-516). The genetic loss of MCL-1 shows a range of phenotypes depending on the developmental timing and tissue deletion. MCL-1 knockout models reveal there are multiple roles for MCL-1 and loss of function impacts a wide range of phenotypes. Global MCL-1-deficient mice display embryonic lethality and studies using conditional genetic deletion have reported mitochondrial dysfunction, impaired activation of autophagy, reductions in B and T lymphocytes, increased B and T cell apoptosis, and the development of heart failure/cardiomyopathy (Wang et al. Genes and Dev 2013; 27 1351-1364, Steimer et al. Blood 2009; (113) 2805-2815).

WO2018178226 discloses MCL-1 inhibitors and methods of use thereof.

WO2017182625 discloses macrocyclic MCL-1 inhibitors for treating cancer.

WO2018178227 discloses the synthesis of MCL-1 inhibitors.

WO2007008627 discloses substituted phenyl derivatives as inhibitors of the activity of anti-apoptotic MCL-1 protein.

WO2008130970 discloses 7-nonsubstituted indole MCL-1 inhibitors.

WO2008131000 discloses 7-substituted indole MCL-1 inhibitors.

WO2020063792 discloses indole macrocyclic derivatives.

WO2020103864 discloses macrocyclic indoles as MCL-1 inhibitors.

WO2020151738 discloses macrocyclic fused pyrrazoles as MCL-1 inhibitors.

WO2020185606 discloses macrocyclic compounds as MCL-1 inhibitors.

There remains a need for MCL-1 inhibitors, useful for the treatment or prevention of cancers such as prostate, lung, pancreatic, breast, ovarian, cervical, melanoma, B-cell chronic lymphocytic leukemia (CLL), acute myeloid leukemia (AML), and acute lymphoblastic leukemia (ALL).

### SUMMARY OF THE INVENTION

The present invention concerns novel compounds of Formula (I):

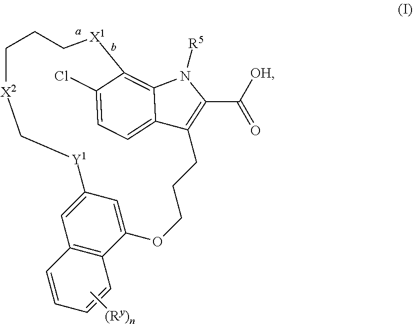

and the tautomers and the stereoisomeric forms thereof, wherein
X¹ represents

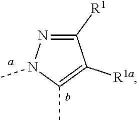

wherein ‘a’ and ‘b’ indicate how variable X¹ is attached to the remainder of the molecule;
R^{y} represents halo;
n represents 0, 1 or 2;
X² represents

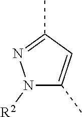

which can be attached to the remainder of the molecule in both directions;
R¹ represents hydrogen; or C₁₋₆alkyl optionally substituted with one substituent selected from the group consisting of Het¹, —OR³, and —NR^{4a}R^{4b};
R² represents hydrogen; methyl; or C₂₋₆alkyl optionally substituted with one substituent selected from the group consisting of Het¹, —OR³, and —NR^{4a}R^{4b};
R^{1a} represents methyl or ethyl;
R³ represents hydrogen, C₁₋₄alkyl, or —C₂₋₄alkyl-O—C₁₋₄alkyl;
R^{4a} and R^{4b} are each independently selected from the group consisting of hydrogen and C₁₋₄alkyl;
R⁵ represents hydrogen; methyl; or C₂₋₆alkyl optionally substituted with one substituent selected from the group consisting of C₃₋₆cycloalkyl, Het¹, —NR^{4a}R^{4b}, and —OR³;
Het¹ represents a 4- to 7-membered monocyclic fully saturated heterocyclyl containing one or two heteroatoms each independently selected from O, S, and N, wherein said S-atom might be substituted to form S(═O) or S(═O)₂; wherein said heterocyclyl is optionally substituted with one or two substituents each independently selected from the group consisting of halo, cyano, and —O—C₁₋₄alkyl;
Y¹ represents —(CH₂)ₘ— or —S—;
m represents 1 or 2;
and the pharmaceutically acceptable salts and the solvates thereof.

The present invention also relates to a pharmaceutical composition comprising a therapeutically effective amount of a compound of Formula (I), a pharmaceutically acceptable salt, or a solvate thereof, and a pharmaceutically acceptable carrier or excipient.

Additionally, the invention relates to a compound of Formula (I), a pharmaceutically acceptable salt, or a solvate thereof, for use as a medicament, and to a compound of Formula (I), a pharmaceutically acceptable salt, or a solvate thereof, for use in the treatment or in the prevention of cancer.

In a particular embodiment, the invention relates to a compound of Formula (I), a pharmaceutically acceptable salt, or a solvate thereof, for use in the treatment or in the prevention of cancer.

The invention also relates to the use of a compound of Formula (I), a pharmaceutically acceptable salt, or a solvate thereof, in combination with an additional pharmaceutical agent for use in the treatment or prevention of cancer.

Furthermore, the invention relates to a process for preparing a pharmaceutical composition according to the invention, characterized in that a pharmaceutically acceptable carrier is intimately mixed with a therapeutically effective amount of a compound of Formula (I), a pharmaceutically acceptable salt, or a solvate thereof.

The invention also relates to a product comprising a compound of Formula (I), a pharmaceutically acceptable salt, or a solvate thereof, and an additional pharmaceutical agent, as a combined preparation for simultaneous, separate or sequential use in the treatment or prevention of cancer.

Additionally, the invention relates to a method of treating or preventing a cell proliferative disease in a subject which comprises administering to the said subject an effective amount of a compound of Formula (I), a pharmaceutically acceptable salt, or a solvate thereof, as defined herein, or a pharmaceutical composition or combination as defined herein.

### DETAILED DESCRIPTION OF THE INVENTION

The term ‘halo’ or ‘halogen’ as used herein represents fluoro, chloro, bromo and iodo.

The prefix ‘C_{x-y}’ (where x and y are integers) as used herein refers to the number of carbon atoms in a given group. Thus, a C₁₋₆alkyl group contains from 1 to 6 carbon atoms, and so on.

The term ‘C₁₋₄alkyl’ as used herein as a group or part of a group represents a straight or branched chain fully saturated hydrocarbon radical having from 1 to 4 carbon atoms, such as methyl, ethyl, n-propyl, isopropyl, n-butyl, s-butyl, t-butyl and the like.

The term ‘C₁₋₆alkyl’ as used herein as a group or part of a group represents a straight or branched chain fully saturated hydrocarbon radical having from 1 to 6 carbon atoms, such as methyl, ethyl, n-propyl, isopropyl, n-butyl, s-butyl, t-butyl, n-pentyl, n-hexyl and the like.

The term ‘C₂₋₄alkyl’ as used herein as a group or part of a group represents a straight or branched chain fully saturated hydrocarbon radical having from 2 to 4 carbon atoms, such as ethyl, n-propyl, isopropyl, n-butyl, s-butyl, t-butyl and the like.

The term ‘C₂₋₆alkyl’ as used herein as a group or part of a group represents a straight or branched chain fully saturated hydrocarbon radical having from 2 to 6 carbon atoms, such as ethyl, n-propyl, isopropyl, n-butyl, s-butyl, t-butyl, n-pentyl, n-hexyl and the like.

The term ‘C₃₋₆cycloalkyl’ as used herein as a group or part of a group defines a fully saturated, cyclic hydrocarbon radical having from 3 to 6 carbon atoms, such as cyclopropyl, cyclobutyl, cyclopentyl and cyclohexyl.

It will be clear for the skilled person that S(═O)₂ or SO₂ represents a sulfonyl moiety.

It will be clear for the skilled person that CO or C(═O) represents a carbonyl moiety.

Non-limiting examples of 4- to 7-membered monocyclic fully saturated heterocyclyl containing one or two heteroatoms each independently selected from O, S, and N, include, but are not limited to tetrahydropyranyl, tetrahydrofuranyl, morpholinyl, 1,4-dioxanyl, oxetanyl, pyrrolidinyl, piperidinyl, piperazinyl, and azetidinyl.

In general, whenever the term ‘substituted’ is used in the present invention, it is meant, unless otherwise indicated or clear from the context, to indicate that one or more hydrogens, in particular from 1 to 4 hydrogens, more in particular from 1 to 3 hydrogens, preferably 1 or 2 hydrogens, more preferably 1 hydrogen, on the atom or radical indicated in the expression using ‘substituted’ are replaced with a selection from the indicated group, provided that the normal valency is not exceeded, and that the substitution results in a chemically stable compound, i.e. a compound that is sufficiently robust to survive isolation to a useful degree of purity from a reaction mixture.

Combinations of substituents and/or variables are permissible only if such combinations result in chemically stable compounds. ‘Stable compound’ is meant to indicate a compound that is sufficiently robust to survive isolation to a useful degree of purity from a reaction mixture.

The skilled person will understand that the term ‘optionally substituted’ means that the atom or radical indicated in the expression using ‘optionally substituted’ may or may not be substituted (this means substituted or unsubstituted respectively).

When two or more substituents are present on a moiety they may, where possible and unless otherwise indicated or clear from the context, replace hydrogens on the same atom or they may replace hydrogen atoms on different atoms in the moiety.

Het¹ may be attached to the remainder of the molecule of Formula (I) through any available ring carbon or nitrogen atom as appropriate, if not otherwise specified.

It will be clear that an alternative presentation (with the structure of X¹ in the Formula) of a Compound of Formula (I) is

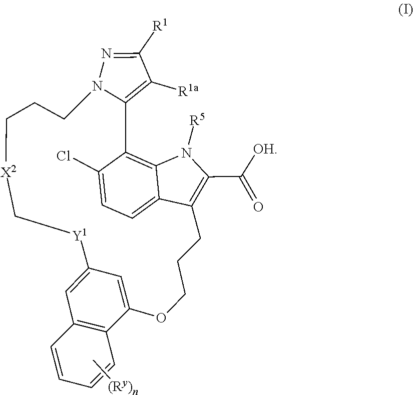

It will be clear that a Compound of Formula (I) includes Compounds of Formula (I-x) and (I-y) (both directions of X² being

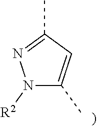

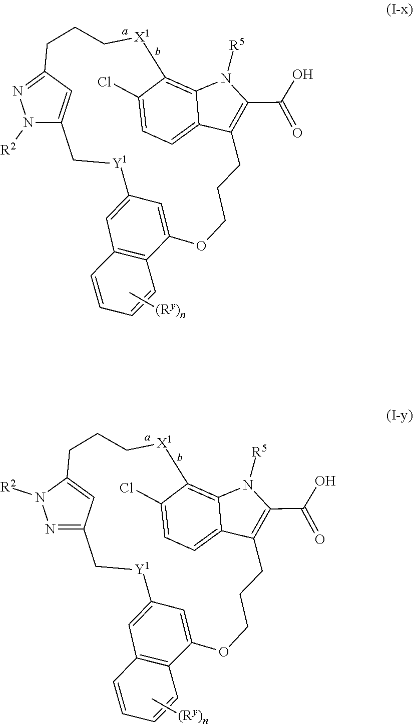

When any variable occurs more than one time in any constituent, each definition is independent.

When any variable occurs more than one time in any formula (e.g. Formula (I)), each definition is independent.

The term “subject” as used herein, refers to an animal, preferably a mammal (e.g. cat, dog, primate or human), more preferably a human, who is or has been the object of treatment, observation or experiment.

The term “therapeutically effective amount” as used herein, means that amount of active compound or pharmaceutical agent that elicits the biological or medicinal response in a tissue system, or subject (e.g., human) that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

The term “treatment”, as used herein, is intended to refer to all processes wherein there may be a slowing, interrupting, arresting or stopping of the progression of a disease, but does not necessarily indicate a total elimination of all symptoms.

The term “compound(s) of the (present) invention” or “compound(s) according to the (present) invention” as used herein, is meant to include the compounds of Formula (I) and the pharmaceutically acceptable salts, and the solvates thereof.

As used herein, any chemical formula with bonds shown only as solid lines and not as solid wedged or hashed wedged bonds, or otherwise indicated as having a particular configuration (e.g. R, S) around one or more atoms, contemplates each possible stereoisomer, or mixture of two or more stereoisomers.

Hereinbefore and hereinafter, the term “compound(s) of Formula (I)” is meant to include the tautomers thereof and the stereoisomeric forms thereof.

The terms “stereoisomers”, “stereoisomeric forms” or “stereochemically isomeric forms” hereinbefore or hereinafter are used interchangeably.

The invention includes all stereoisomers of the compounds of the invention either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Atropisomers (or atropoisomers) are stereoisomers which have a particular spatial configuration, resulting from a restricted rotation about a single bond, due to large steric hindrance. All atropisomeric forms of the compounds of Formula (I) are intended to be included within the scope of the present invention.

In particular, the compounds disclosed herein possess axial chirality, by virtue of restricted rotation around a biaryl bond and as such may exist as mixtures of atropisomers. When a compound is a pure atropisomer, the stereochemistry at each chiral center may be specified by either Rₐ or Sₐ. Such designations may also be used for mixtures that are enriched in one atropisomer. Further description of atropisomerism and axial chirality and rules for assignment of configuration can be found in Eliel, E. L. & Wilen, S. H. ‘Stereochemistry of Organic Compounds’ John Wiley and Sons, Inc. 1994.

Diastereomers (or diastereoisomers) are stereoisomers that are not enantiomers, i.e. they are not related as mirror images. If a compound contains a double bond, the substituents may be in the E or the Z configuration.

Substituents on bivalent cyclic saturated or partially saturated radicals may have either the cis- or trans-configuration; for example if a compound contains a disubstituted cycloalkyl group, the substituents may be in the cis or trans configuration.

Therefore, the invention includes enantiomers, atropisomers, diastereomers, racemates, E isomers, Z isomers, cis isomers, trans isomers and mixtures thereof, whenever chemically possible.

The meaning of all those terms, i.e. enantiomers, atropisomers, diastereomers, racemates, E isomers, Z isomers, cis isomers, trans isomers and mixtures thereof are known to the skilled person.

The absolute configuration is specified according to the Cahn-Ingold-Prelog system. The configuration at an asymmetric atom is specified by either R or S. Resolved stereoisomers whose absolute configuration is not known can be designated by (+) or (−) depending on the direction in which they rotate plane polarized light. For instance, resolved enantiomers whose absolute configuration is not known can be designated by (+) or (−) depending on the direction in which they rotate plane polarized light. Optically active (Rₐ)- and (Sₐ)-atropisomers may be prepared using chiral synthons, chiral reagents or chiral catalysts, or resolved using conventional techniques well known in the art, such as chiral HPLC.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other stereoisomers. Thus, when a compound of Formula (I) is for instance specified as (R), this means that the compound is substantially free of the (S) isomer; when a compound of Formula (I) is for instance specified as E, this means that the compound is substantially free of the Z isomer; when a compound of Formula (I) is for instance specified as cis, this means that the compound is substantially free of the trans isomer; when a compound of Formula (I) is for instance specified as Rₐ, this means that the compound is substantially free of the Sₐ atropisomer.

Pharmaceutically acceptable salts, in particular pharmaceutically acceptable additions salts, include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

The pharmaceutically acceptable salts as mentioned hereinabove or hereinafter are meant to comprise the therapeutically active non-toxic acid and base salt forms which the compounds of Formula (I), and solvates thereof, are able to form.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

The compounds of Formula (I) and solvates thereof containing an acidic proton may also be converted into their non-toxic metal or amine salt forms by treatment with appropriate organic and inorganic bases.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the solvent addition forms as well as the salts thereof, which the compounds of Formula (I) are able to form. Examples of such solvent addition forms are e.g. hydrates, alcoholates and the like.

The compounds of the invention as prepared in the processes described below may be synthesized in the form of mixtures of enantiomers, in particular racemic mixtures of enantiomers, that can be separated from one another following art-known resolution procedures. A manner of separating the enantiomeric forms of the compounds of Formula (I), and pharmaceutically acceptable salts, and solvates thereof, involves liquid chromatography using a chiral stationary phase. Said pure stereochemically isomeric forms may also be derived from the corresponding pure stereochemically isomeric forms of the appropriate starting materials, provided that the reaction occurs stereospecifically. Preferably if a specific stereoisomer is desired, said compound would be synthesized by stereospecific methods of preparation. These methods will advantageously employ enantiomerically pure starting materials.

The term “enantiomerically pure” as used herein means that the product contains at least 80% by weight of one enantiomer and 20% by weight or less of the other enantiomer. Preferably the product contains at least 90% by weight of one enantiomer and 10% by weight or less of the other enantiomer. In the most preferred embodiment the term “enantiomerically pure” means that the composition contains at least 99% by weight of one enantiomer and 1% or less of the other enantiomer.

The present invention also embraces isotopically-labeled compounds of the present invention which are identical to those recited herein, but for the fact that one or more atoms are replaced by an atom having an atomic mass or mass number different from the atomic mass or mass number usually found in nature (or the most abundant one found in nature).

All isotopes and isotopic mixtures of any particular atom or element as specified herein are contemplated within the scope of the compounds of the invention, either naturally occurring or synthetically produced, either with natural abundance or in an isotopically enriched form. Exemplary isotopes that can be incorporated into compounds of the invention include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵O, ¹⁷O, ¹⁸O, ³²P, ³³P, ³⁵S ¹⁸F, ³⁶Cl, ¹²²I, ¹²³I, ¹²⁵I, ¹³¹I, ⁷⁵Br, ⁷⁶Br, ⁷⁷Br and ⁸²Br. Preferably, the isotope is selected from the group of ²H, ³H, ¹¹C and ¹⁸F. More preferably, the isotope is ²H. In particular, deuterated compounds are intended to be included within the scope of the present invention.

Certain isotopically-labeled compounds of the present invention (e.g., those labeled with ³H and ¹⁴C) may be useful for example in substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies. PET imaging in cancer finds utility in helping locate and identify tumours, stage the disease and determine suitable treatment. Human cancer cells overexpress many receptors or proteins that are potential disease-specific molecular targets. Radiolabelled tracers that bind with high affinity and specificity to such receptors or proteins on tumour cells have great potential for diagnostic imaging and targeted radionuclide therapy (Charron, Carlie L. et al. Tetrahedron Lett. 2016, 57(37), 4119-4127). Additionally, target-specific PET radiotracers may be used as biomarkers to examine and evaluate pathology, by for example, measuring target expression and treatment response (Austin R. et al. Cancer Letters (2016), doi: 10.1016/j.canlet.2016.05.008).

The present invention relates in particular to compounds of Formula (I) as defined herein, and the tautomers and the stereoisomeric forms thereof, wherein

X¹ represents

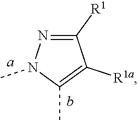

wherein ‘a’ and ‘b’ indicate how variable X¹ is attached to the remainder of the molecule;
R^{y} represents halo;
n represents 0 or 1;
X² represents

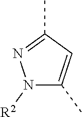

which can be attached to the remainder of the molecule in both directions;
R¹ represents methyl;
R² represents methyl; or C₂₋₆alkyl optionally substituted with one substituent selected from the group consisting of —OR³, and —NR^{4a}R^{4b};
R^{1a} represents methyl;
R³ represents C₁₋₄alkyl or —C₂₋₄alkyl-O—C₁₋₄alkyl;
R^{4a} and R^{4b} are each independently selected from the group consisting of hydrogen and C₁₋₄alkyl;
R⁵ represents methyl; or C₂₋₆alkyl optionally substituted with one —OR³;
Y¹ represents —(CH₂)ₘ— or —S—;
m represents 1.

The present invention relates in particular to compounds of Formula (I) as defined herein, and the tautomers and the stereoisomeric forms thereof, wherein

X¹ represents

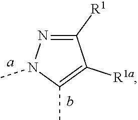

wherein ‘a’ and ‘b’ indicate how variable X¹ is attached to the remainder of the molecule;
R^{y} represents fluoro;
n represents 1;
X² represents

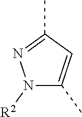

which can be attached to the remainder of the molecule in both directions;
R¹ represents methyl;
R² represents methyl; or C₂₋₆alkyl optionally substituted with one substituent selected from the group consisting of —OR³, and —NR^{4a}R^{4b};
R^{1a} represents methyl;
R³ represents C₁₋₄alkyl or —C₂₋₄alkyl-O—C₁₋₄alkyl; R^{4a} and R^{4b} are each independently selected from the group consisting of hydrogen and C₁₋₄alkyl;
R⁵ represents methyl; or C₂₋₆alkyl optionally substituted with one —OR³;
Y¹ represents —(CH₂)ₘ— or —S—;
m represents 1.

The present invention relates in particular to compounds of Formula (I) as defined herein, and the tautomers and the stereoisomeric forms thereof, wherein X¹ represents

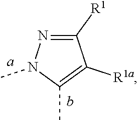

wherein ‘a’ and ‘b’ indicate how variable X¹ is attached to the remainder of the molecule;
R^{y} represents halo;
n represents 0 or 1;
X² represents

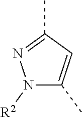

which can be attached to the remainder of the molecule in both directions;
R¹ represents methyl;
R² represents methyl;
R^{1a} represents methyl;
R³ represents —C₂₋₄alkyl-O—C₁₋₄alkyl;
R⁵ represents methyl; or C₂₋₆alkyl optionally substituted with one —OR³;
Y¹ represents —(CH₂)ₘ— or —S—;
m represents 1.

The present invention relates in particular to compounds of Formula (I) as defined herein, and the tautomers and the stereoisomeric forms thereof, wherein

X¹ represents

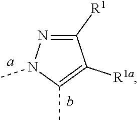

wherein ‘a’ and ‘b’ indicate how variable X¹ is attached to the remainder of the molecule;
R^{y} represents fluoro;
n represents 1;
X² represents

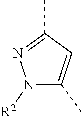

which can be attached to the remainder of the molecule in both directions;
R¹ represents methyl;
R² represents methyl;
R^{1a} represents methyl;
R³ represents —C₂₋₄alkyl-O—C₁₋₄alkyl;
R⁵ represents methyl; or C₂₋₆alkyl optionally substituted with one —OR³;
Y¹ represents —(CH₂)ₘ— or —S—;
m represents 1.

The present invention relates in particular to compounds of Formula (I) as defined herein, and the tautomers and the stereoisomeric forms thereof, wherein

X¹ represents

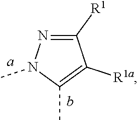

wherein ‘a’ and ‘b’ indicate how variable X¹ is attached to the remainder of the molecule;
R^{y} represents halo;
n represents 0 or 1;
X² represents

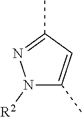

which can be attached to the remainder of the molecule in both directions;
R¹ represents methyl;
R² represents methyl;
R^{1a} represents methyl;
R⁵ represents methyl; or C₂₋₆alkyl optionally substituted with one —OR³;
R³ represents —C₂₋₄alkyl-O—C₁₋₄alkyl;
Y¹ represents —S—;
and the pharmaceutically acceptable salts and the solvates thereof.

The present invention relates in particular to compounds of Formula (I) as defined herein, and the tautomers and the stereoisomeric forms thereof, wherein

X¹ represents

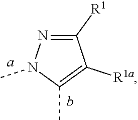

wherein ‘a’ and ‘b’ indicate how variable X¹ is attached to the remainder of the molecule;
R^{y} represents fluoro;
n represents 1;
X² represents

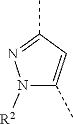

which can be attached to the remainder of the molecule in both directions;
R¹ represents methyl;
R² represents methyl;
R^{1a} represents methyl;
R⁵ represents C₂₋₆alkyl optionally substituted with one —OR³;
R³ represents —C₂₋₄alkyl-O—C₁₋₄alkyl;
Y¹ represents —S—;
and the pharmaceutically acceptable salts and the solvates thereof.

The present invention relates in particular to compounds of Formula (I) as defined herein, and the tautomers and the stereoisomeric forms thereof, wherein

X¹ represents

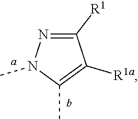

wherein ‘a’ and ‘b’ indicate how variable X¹ is attached to the remainder of the molecule;
n represents 0;
X² represents

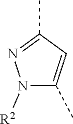

which can be attached to the remainder of the molecule in both directions;
R¹ represents methyl;
R² represents methyl;
R^{1a} represents methyl;
R⁵ represents methyl; or C₂₋₆alkyl optionally substituted with one —OR³;
R³ represents —C₂₋₄alkyl-O—C₁₋₄alkyl;
Y¹ represents —S—;
and the pharmaceutically acceptable salts and the solvates thereof.

In an embodiment, the present invention relates to those compounds of Formula (I) and the pharmaceutically acceptable salts, and the solvates thereof, or any subgroup thereof as mentioned in any of the other embodiments, wherein Y¹ represents —S—.

In an embodiment, the present invention relates to those compounds of Formula (I) and the pharmaceutically acceptable salts, and the solvates thereof, or any subgroup thereof as mentioned in any of the other embodiments, wherein R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein

n represents 1; and
R^{y} represents fluoro.

In an embodiment, the present invention relates to those compounds of Formula (I) and the pharmaceutically acceptable salts, and the solvates thereof, or any subgroup thereof as mentioned in any of the other embodiments, wherein R¹ represents hydrogen.

In an embodiment, the present invention relates to those compounds of Formula (I) and the pharmaceutically acceptable salts, and the solvates thereof, or any subgroup thereof as mentioned in any of the other embodiments, wherein R¹ represents methyl.

In an embodiment, the present invention relates to those compounds of Formula (I) and the pharmaceutically acceptable salts, and the solvates thereof, or any subgroup thereof as mentioned in any of the other embodiments, wherein R² represents methyl.

In an embodiment, the present invention relates to those compounds of Formula (I) and the pharmaceutically acceptable salts, and the solvates thereof, or any subgroup thereof as mentioned in any of the other embodiments, wherein R⁵ represents methyl.

In an embodiment, the present invention relates to those compounds of Formula (I) and the pharmaceutically acceptable salts, and the solvates thereof, or any subgroup thereof as mentioned in any of the other embodiments, wherein R⁵ represents ethyl.

In an embodiment, the present invention relates to those compounds of Formula (I) and the pharmaceutically acceptable salts, and the solvates thereof, or any subgroup thereof as mentioned in any of the other embodiments, wherein

R⁵ represents methyl; or C₂₋₆alkyl optionally substituted with C₃₋₆cycloalkyl or Het¹.

In an embodiment, the present invention relates to those compounds of Formula (I) and the pharmaceutically acceptable salts, and the solvates thereof, or any subgroup thereof as mentioned in any of the other embodiments, wherein

R⁵ represents methyl; or C₂₋₆alkyl optionally substituted with one —OR³.

In an embodiment, the present invention relates to those compounds of Formula (I) and the pharmaceutically acceptable salts, and the solvates thereof, or any subgroup thereof as mentioned in any of the other embodiments, wherein

R⁵ represents C₂₋₆alkyl optionally substituted with one —OR³.

In an embodiment, the present invention relates to those compounds of Formula (I) and the pharmaceutically acceptable salts, and the solvates thereof, or any subgroup thereof as mentioned in any of the other embodiments, wherein

R⁵ represents C₂₋₆alkyl optionally substituted with one —OR³; and
R³ represents —C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein

R⁵ represents C₂₋₆alkyl substituted with one —OR³; and
R³ represents —C₂₋₄alkyl-O—C₁₋₄alkyl.

In an embodiment, the present invention relates to those compounds of Formula (I) and the pharmaceutically acceptable salts, and the solvates thereof, or any subgroup thereof as mentioned in any of the other embodiments, wherein n represents 0.

In an embodiment, the present invention relates to those compounds of Formula (I) and the pharmaceutically acceptable salts, and the solvates thereof, or any subgroup thereof as mentioned in any of the other embodiments, wherein n represents 1.

In an embodiment, the present invention relates to those compounds of Formula (I) and the pharmaceutically acceptable salts, and the solvates thereof, or any subgroup thereof as mentioned in any of the other embodiments, wherein n represents 2.

In an embodiment, the present invention relates to those compounds of Formula (I) and the pharmaceutically acceptable salts, and the solvates thereof, or any subgroup thereof as mentioned in any of the other embodiments, wherein m represents 1.

In an embodiment, the present invention relates to those compounds of Formula (I) and the pharmaceutically acceptable salts, and the solvates thereof, or any subgroup thereof as mentioned in any of the other embodiments, wherein m represents 2.

In an embodiment, the present invention relates to those compounds of Formula (I) and the pharmaceutically acceptable salts, and the solvates thereof, or any subgroup thereof as mentioned in any of the other embodiments, wherein Het¹ is attached to the remainder of the molecule of Formula (I) through a nitrogen atom.

In an embodiment, the present invention relates to those compounds of Formula (I) and the pharmaceutically acceptable salts, and the solvates thereof, or any subgroup thereof as mentioned in any of the other embodiments, wherein n is 1 and wherein R^{y} is in position 3 as indicated below:

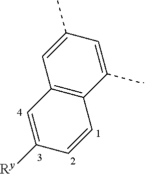

In an embodiment, the present invention relates to those compounds of Formula (I) and the pharmaceutically acceptable salts, and the solvates thereof, or any subgroup thereof as mentioned in any of the other embodiments, wherein n is 1 and wherein R^{y} is in position 3 as indicated below; and wherein R^{y} represents fluoro:

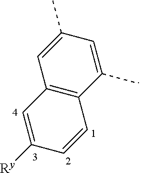

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-x):

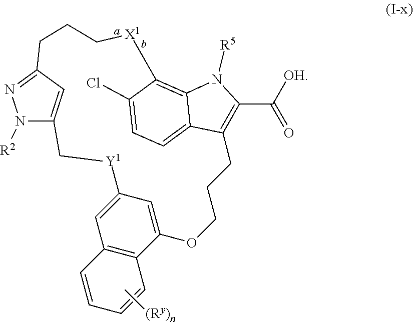

It will be clear that all variables in the structure of Formula (I-x), are defined as defined for the compounds of Formula (I) or any subgroup thereof as mentioned in any of the other embodiments.

In an embodiment, the present invention relates to those compounds of Formula (I) and the pharmaceutically acceptable salts, and the solvates thereof, or any subgroup thereof as mentioned in any of the other embodiments, wherein the compounds of Formula (I) are restricted to compounds of Formula (I-y):

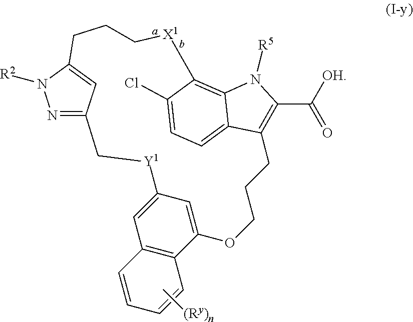

It will be clear that all variables in the structure of Formula (I-y), are defined as defined for the compounds of Formula (I) or any subgroup thereof as mentioned in any of the other embodiments.

In an embodiment, the present invention relates to a subgroup of Formula (I) as defined in the general reaction schemes.

In an embodiment the compound of Formula (I) is selected from the group consisting of any of the exemplified compounds, tautomers and stereoisomeric forms thereof, any pharmaceutically acceptable salts, and the solvates thereof.

All possible combinations of the above indicated embodiments are considered to be embraced within the scope of the invention.

### Methods for the Preparation of Compounds

In this section, as in all other sections unless the context indicates otherwise, references to Formula (I) also include all other sub-groups and examples thereof as defined herein.

The general preparation of some typical examples of the compounds of Formula (I) is described hereunder and in the specific examples, and are generally prepared from starting materials which are either commercially available or prepared by standard synthetic processes commonly used by those skilled in the art of organic chemistry. The following schemes are only meant to represent examples of the invention and are in no way meant to be a limit of the invention.

Alternatively, compounds of the present invention may also be prepared by analogous reaction protocols as described in the general schemes below, combined with standard synthetic processes commonly used by those skilled in the art.

The skilled person will realize that in the reactions described in the Schemes, although this is not always explicitly shown, it may be necessary to protect reactive functional groups (for example hydroxy, amino, or carboxy groups) where these are desired in the final product, to avoid their unwanted participation in the reactions. In general, conventional protecting groups can be used in accordance with standard practice. The protecting groups may be removed at a convenient subsequent stage using methods known from the art.

The skilled person will realize that in the reactions described in the Schemes, it may be advisable or necessary to perform the reaction under an inert atmosphere, such as for example under N₂-gas atmosphere.

It will be apparent for the skilled person that it may be necessary to cool the reaction mixture before reaction work-up (refers to the series of manipulations required to isolate and purify the product(s) of a chemical reaction such as for example quenching, column chromatography, extraction).

The skilled person will realize that heating the reaction mixture under stirring may enhance the reaction outcome. In some reactions microwave heating may be used instead of conventional heating to shorten the overall reaction time.

The skilled person will realize that another sequence of the chemical reactions shown in the Schemes below, may also result in the desired compound of Formula (I).

The skilled person will realize that intermediates and final compounds shown in the Schemes below may be further functionalized according to methods well-known by the person skilled in the art. The intermediates and compounds described herein can be isolated in free form or as a salt, or a solvate thereof. The intermediates and compounds described herein may be synthesized in the form of mixtures of tautomers and stereoisomeric forms that can be separated from one another following art-known resolution procedures.

Compounds of Formula (I) can be prepared according to Scheme 1,

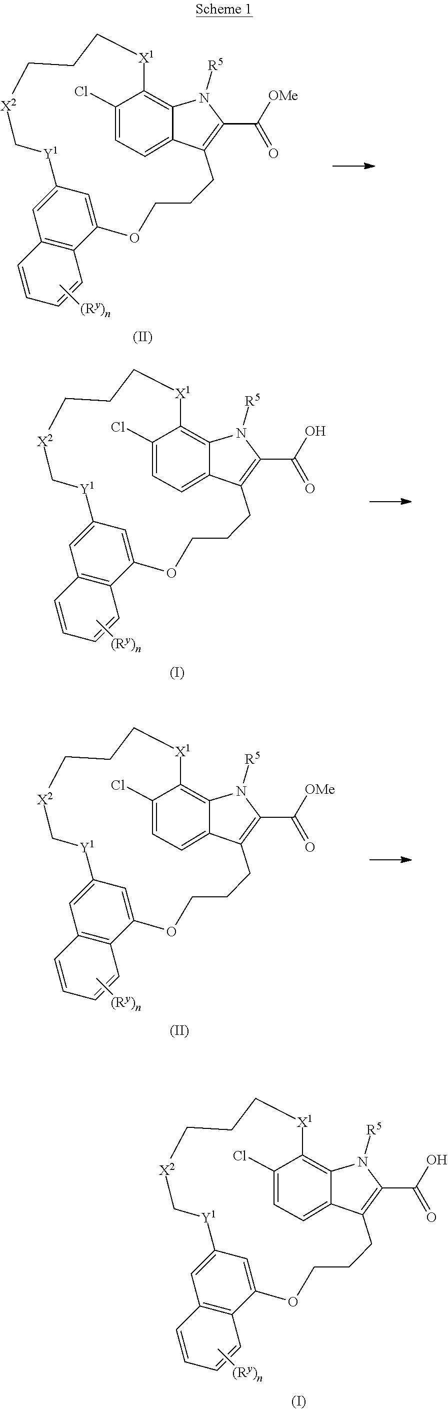

by reacting an intermediate of Formula (II), where X¹, X², Y¹, R⁵, and (R^{y})ₙ are defined as in Formula (I), with a suitable base such as, for example, LiOH or NaOH, in a suitable solvent such as, for example, water or a mixture of water and a suitable organic solvent such as dioxane or THF, or a mixture of MeOH and THF, at a suitable temperature such as room temperature or 60° C.
Intermediates of Formula (II) can be prepared by reacting an intermediate of Formula (III) with a suitable reagent, such as, for example, diethyl azodicarboxylate (DEAD) or di-tert-butyl azodicarboxylate (DTBAD), in the presence of a suitable phosphine such as, for example, triphenylphosphine (PPh₃), in a suitable solvent such as, for example, THF, toluene, or a mixture thereof, at a suitable temperature such as, for example, room temperature or 70° C.
Intermediates of Formula (III) can be prepared by reacting an intermediate of Formula (IV), wherein Y² is C═O and R′ is Me, with a suitable reducing agent such as, for example, BH₃.DMS (borane dimethylsulfide), in a suitable solvent such as, for example, THF, at a suitable temperature such as, for example, room temperature or 50° C.
Alternatively, intermediates of Formula (III) can be prepared by reacting an intermediate of Formula (IV), wherein Y² is CH₂ and R′ is a suitable protecting group such as tert-butyldimethylsilyl (TBDMS), with a suitable deprotecting agent such as, for example, tetrabutylammonium fluoride (TBAF), in a suitable solvent such as, for example, THF, at a suitable temperature such as, for example, room temperature.

A skilled person will understand that this whole synthetic pathway can be followed with R⁵ being H (hydrogen). In such a case, R⁵ (in that case, defined as in Formula (I)) can be introduced by reacting an intermediate of Formula (II), wherein R⁵ is hydrogen, with a suitable alkylating agent R⁵L, wherein L is a suitable leaving group, such as, for example, 1-bromo-2-(2-methoxyethoxy)ethane, in the presence of a suitable base such as, for example, Cs₂CO₃, in a suitable solvent such as, for example, DMF, at a suitable temperature such as, for example, 50° C.

Compounds of Formula (IV), wherein Y¹ is defined as S (sulfur), and X¹, X², R⁵, and (R^{y})ₙ are defined as in Formula (I), Hal is defined as a suitable halogen, and Y²/R′ is C═O/Me or Y²/R′ is CH₂/TBDMS, can be prepared according to Scheme 2,

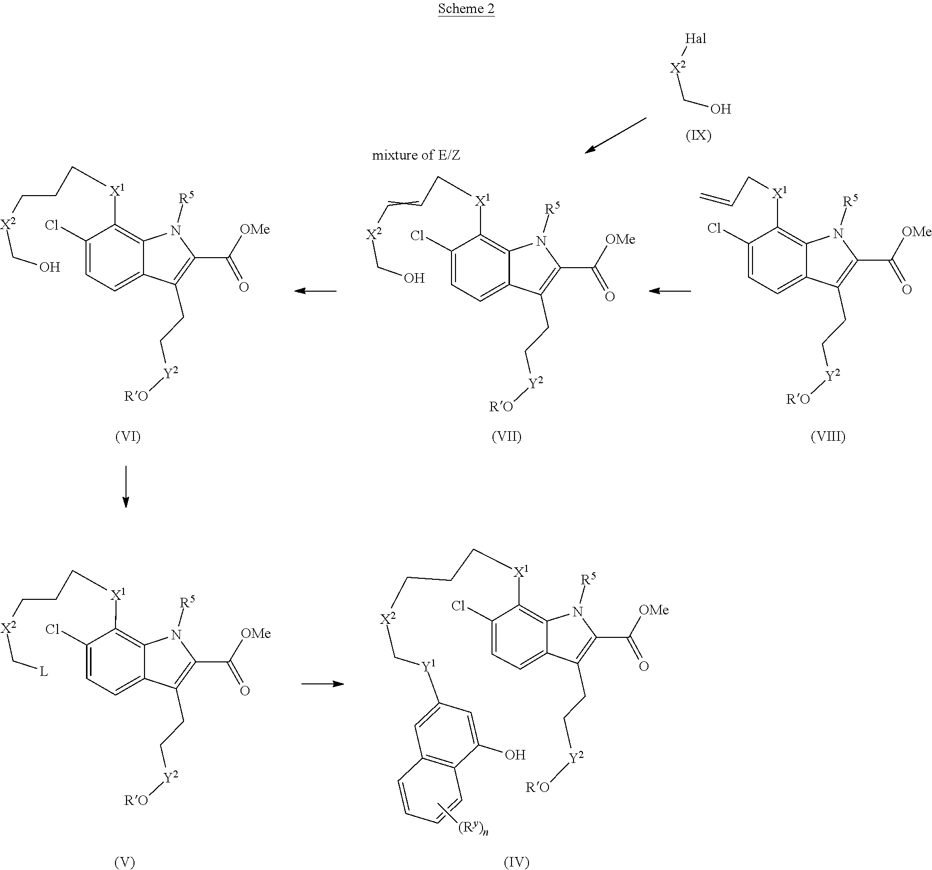

by reacting an intermediate of Formula (V) with a suitable acetyloxy-naphthalenyl thioester such as, for example, ethanethioic acid, S-[4-(acetyloxy)-2-naphthalenyl] ester (CAS [2143010-96-0]), in the presence of a suitable base such as, for example, K₂CO₃, in a suitable solvent such as, for example, MeOH or THF, or a mixture thereof, at a suitable temperature such as, for example, room temperature.
Intermediates of Formula (V) can be prepared by reacting an intermediate of Formula (VI) with a suitable leaving group precursor such as, for example, MsCl, in the presence of a suitable base such as, for example, Et₃N, in a suitable solvent such as, for example, DCM, at a suitable temperature such as, for example, 0° C. or room temperature.
Intermediates of Formula (VI) can be prepared by reacting an intermediate of Formula (VII) with a suitable hydrogenating agent such as, for example, hydrogen, in the presence of a suitable catalyst such as, for example, Pd/C, in a suitable solvent such as, for example, EtOAc, at a suitable temperature such as, for example, room temperature.
Intermediates of Formula (VII) can be prepared by reacting an intermediate of Formula (VIII) with an intermediate of Formula (IX), in the presence of a suitable base such as, for example, di-isopropylethylamine (DIPEA), and a suitable catalyst such as, for example, bis(tri-tert-butylphosphine)palladium(0) (Pd(tBu₃P)₂), in a suitable solvent such as, for example, N,N-dimethylformamide (DMF), at a suitable temperature such as, for example, 120° C.

Intermediates of Formula (VIII) where X¹, R⁵, and (R^{y})ₙ are defined as in Formula (I), and Y²/R′ is C═O/Me or Y²/R′ is CH₂/TBDMS, can be prepared according to Scheme 3,

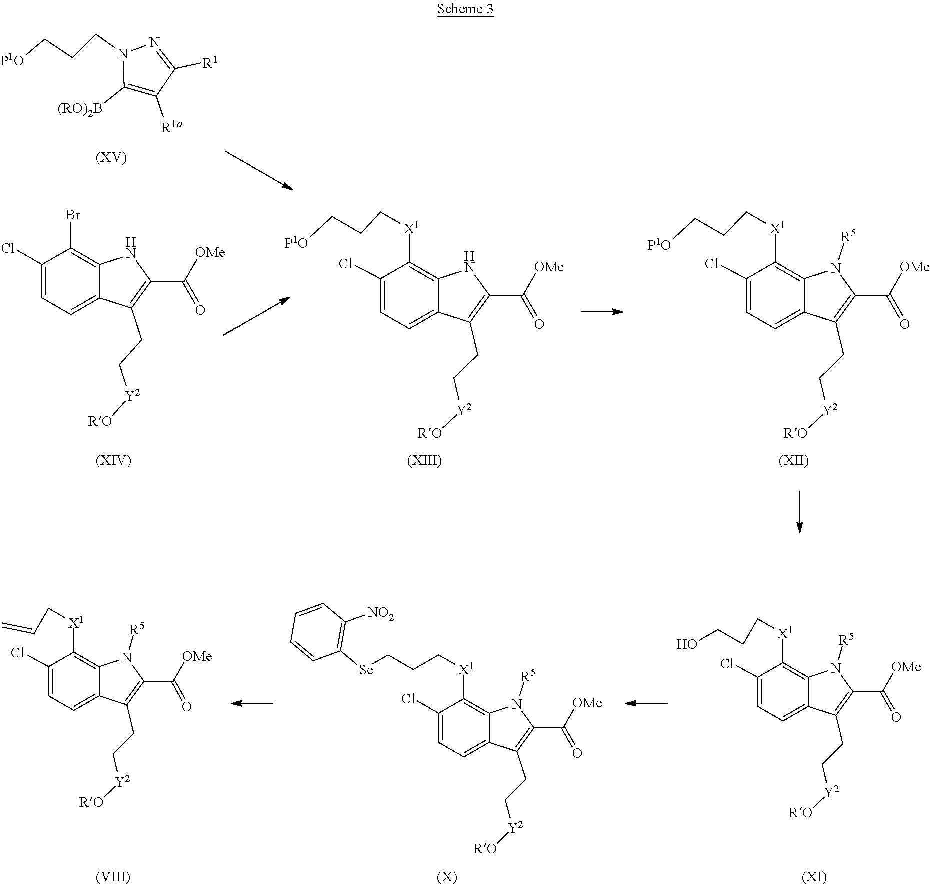

by reacting an intermediate of Formula (X) with a suitable oxidizing agent such as, for example, hydrogen peroxide, in a suitable solvent such as, for example, THF, at a suitable temperature such as, for example, room temperature.
Intermediates of Formula (X) can be prepared by reacting an intermediate of Formula (XI) with a suitable dehydration reagent such as, for example, 2-nitrophenyl selenocyanate, in the presence of a suitable activating agent such as, for example, tri-n-butylphosphine, in a suitable solvent such as, for example, THF, at a suitable temperature such as, for example, room temperature.
Intermediates of Formula (XI) can be prepared by reacting an intermediate of Formula (XII), wherein P¹ is a suitable protecting groups such as, for example, TBDMS, with a suitable deprotecting agent such as, for example, TBAF, in a suitable solvent such as, for example, THF, at a suitable temperature such as, for example, room temperature.
It will be clear to a skilled person, that for the synthesis of intermediates of Formula (XI) orthogonality of protective groups will need to be respected between P¹ and R′ when Y² is CH₂.
Intermediates of Formula (XII) can be prepared by reacting an intermediate of Formula (XIII) with a suitable alkylating agent such as, for example, methyl iodide, in the presence of a suitable base such as, for example Cs₂CO₃, in a suitable solvent such as, for example, DMF, at a suitable temperature such as, for example, room temperature.
Intermediates of Formula (XIII) can be prepared by reacting an intermediate of Formula (XIV), wherein Y² is C═O and R′=Me (methyl 7-bromo-6-chloro-3-(3-methoxy-3-oxopropyl)-1H-indole-2-carboxylate, CAS [2143010-85-7]) with an intermediate of Formula (XV), in which R¹ and R^{1a} are as defined in Formula (I), in the presence of a suitable base such as, for example, K₂CO₃, and a suitable catalyst such as, for example, bis(di-tert-butyl(4-dimethylaminophenyl)phosphine) (Pd(amphos)Cl₂) (CAS [887919-35-9]), in a suitable solvent such as, for example, a mixture of 1,4-dioxane and water, at a suitable temperature such as, for example, 80° C.
Alternatively, this whole synthetic pathway may start from methyl 7-bromo-6-chloro-3-(3-hydroxypropyl)-1H-indole-2-carboxylate (CAS [2245716-18-9]) after its protection by a suitable protecting group reagent such as, for example, TBDMSCl (tert-butyldimethylchlorosilane), in the presence of a suitable base such as, for example, Et₃N (triethylamine) or DMAP (4-dimethylaminopyridine), or a mixture thereof, in a suitable solvent such as, for example, THF, at a suitable temperature such as, for example, room temperature, leading to intermediates wherein Y² is CH₂ and R′ is a suitable protecting group such as TBDMS.

Intermediates of Formula (XV) where R¹, R^{1a} are defined as in Formula (I), (RO)₂ is a suitable ester group such as, for example, pinacolato, and P¹ is a suitable protective group such as, for example, TBDMS, can be prepared according to Scheme 4,

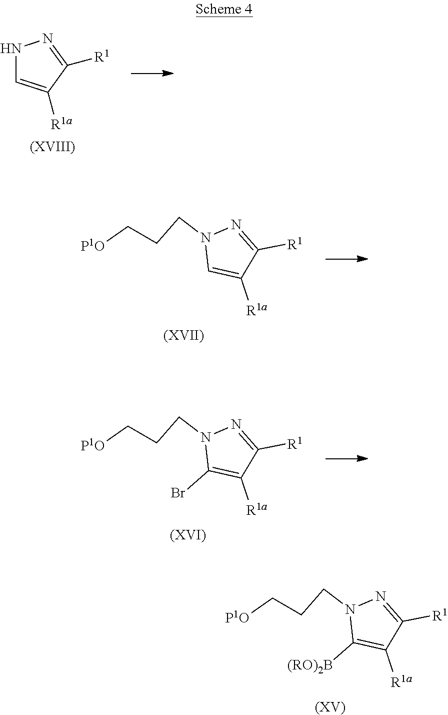

by reacting an intermediate of Formula (XVI) with a suitable boronate precursor such as, for example, 2-isopropoxy-4,4,5,5-tetramethyl-1,3,2-dioxaborolane (CAS [61676-62-8]), in the presence of a suitable base such as, for example, butyl lithium (BuLi), in a suitable solvent such as, for example, tetrahydrofuran (THF), at a suitable temperature such as, for example, −78° C. or room temperature.
Intermediates of Formula (XVI) can be prepared by reacting an intermediate of Formula (XVII) with a suitable brominating agent such as, for example, N-bromosuccinimide, in a suitable solvent such as, for example, DCM, at a suitable temperature such as, for example, 0° C. or room temperature.
Intermediates of Formula (XVII) can be prepared by reacting a suitable pyrazole (XVIII) such as, for example, 3,4-dimethyl-1H-pyrazole [2820-37-3], with a suitable protected alkylating agent such as, for example, (3-bromopropoxy)-tert-butyldimethylsilane (CAS [89031-84-5]), in the presence of a suitable base such as, for example, NaH, in a suitable solvent such as, for example, DMF, at a suitable temperature such as, for example, 0° C. or room temperature.
It will be clear for a skilled person that a mixture of isomers can be obtained during the synthesis of an intermediate of Formula (XVII), of which the desired isomer can be separated using a suitable method such as, for instance, column chromatography.

Intermediates of Formula (IX), wherein R² is defined as in Formula (I), Hal is defined as a suitable halogen such as, for example, Br, and X² is a suitably substituted pyrazolyl group, can be prepared according to Scheme 5,

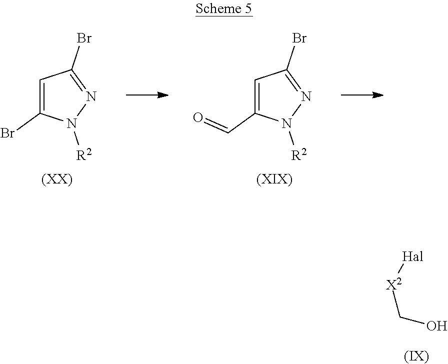

by reacting an intermediate of Formula (XIX) with a suitable reducing agent such as, for example, NaBH₄, in a suitable solvent such as, for example, MeOH, at a suitable temperature such as, for example, 15° C.
Intermediates of Formula (XIX) can be prepared by reacting an intermediate of Formula (XX) with a suitable acylating agent such as, for example, DMF, in the presence of a suitable base such as, for example, buyl lithium (BuLi), in a suitable solvent such as, for example, tetrahydrofuran (THF), at a suitable temperature such as, for example, −78° C.
Intermediates of Formula (XX) are commercially available or can be prepared according to procedures described in literature.

Compounds of Formula (IV), wherein X¹, X², R⁵, and (R^{y})ₙ are defined as in Formula (I), Hal is defined as a suitable halogen, and Y²/R′ is C═O/Me or Y²/R′ is CH₂/TBDMS, Y¹ is defined as (CH₂)ₘ, and m is defined as in Formula (I), can be prepared according to Scheme 6,

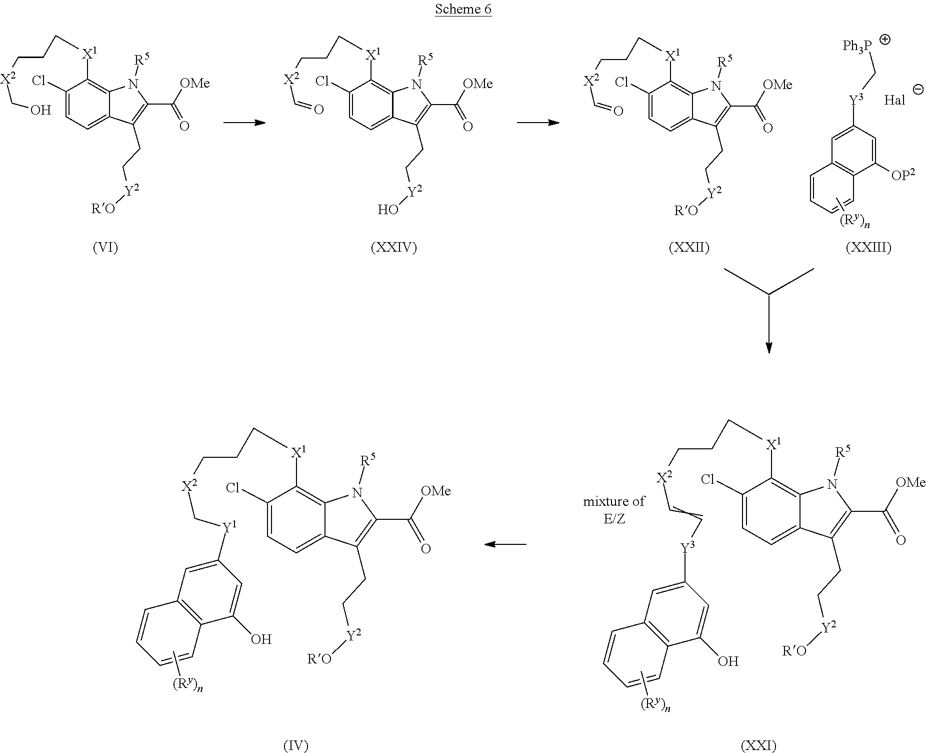

by reacting an intermediate of Formula (XXI), wherein Y³ is a direct bond or CH₂, with a suitable hydrogenating agent such as, for example, hydrogen gas, in the presence of a suitable catalyst such as, for example, Pd/C, in a suitable solvent such as, for example, ethyl acetate (EtOAc), at a suitable temperature such as, for example, room temperature.
Intermediates of Formula (XXI) can be prepared by reacting an intermediate of Formula (XXII) with an intermediate of Formula (XXIII), wherein Y³ is a direct bond or CH₂, in the presence of a suitable base such as, for example, NaH, in a suitable solvent such as, for example, THF, at a suitable temperature such as, for example, room temperature.
Intermediates of Formula (XXII) can be prepared by reacting an intermediate of Formula (XXIV) with a suitable protecting group precursor such as, for example, TBDMSCl, in the presence of a suitable base such as, for example, imidazole, in a suitable solvent such as, for example, DCM, at a suitable temperature such as, for example, room temperature.
Intermediates of Formula (XXIV) can be prepared by reacting an intermediate of Formula (VI) with a suitable oxidizing agent such as, for example, MnO₂, in a suitable solvent such as, for example, DCM, at a suitable temperature such as, for example, reflux of the solvent.

Intermediates of Formula (XXIII) wherein Y³ is a direct bond or CH₂, R^{y} and n are defined as in Formula (I), Hal is defined as a suitable halogen, and P² is a suitable protecting group such as, for example, tert-butyldiphenylsilyl (TBDPS), can be prepared according to Scheme 7,

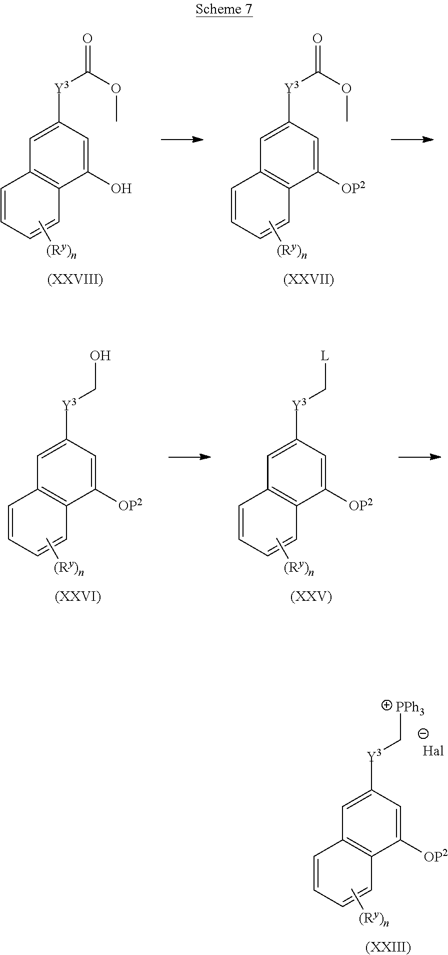

by reacting an intermediate of Formula (XXV), wherein L is a suitable leaving group such as, for example, a mesylate, with a suitable phosphine such as, for example, PPh₃, in a suitable solvent such as, for example, acetonitrile, at a suitable temperature such as, for example, 85° C.
Intermediates of Formula (XXV) can be prepared by reacting an intermediate of Formula (XXVI) with a suitable activating agent such as, for example, mesyl chloride (MsCl), in the presence of a suitable base such as, for example, DIPEA, in a suitable solvent such as, for example, DCM, at a suitable temperature such as, for example, room temperature.
Intermediates of Formula (XXVI) can be prepared by reacting an intermediate of Formula (XXVII), with a suitable reducing agent such as, for example, LiAlH₄, in a suitable solvent such as, for example, THF, at a suitable temperature such as, for example, 0° C.
Intermediates of Formula (XXVII) can be prepared by reacting an intermediate of Formula (XXVIII) with a suitable protecting group such as, for example, TBDPSCl, in the presence of a suitable base such as, for example, imidazole, in a suitable solvent such as, for example, DMF, at a suitable temperature such as, for example, room temperature.
Intermediates of Formula (XXVIII) are commercially available or can be prepared in analogy with procedures described in literature.

Alternatively, Intermediates of Formula (IV), wherein X¹, X², R⁵, and (R^{y})ₙ are defined as in Formula (I), and Y²/R′ is C═O/Me or Y²/R′ is CH₂/TBDMS, Hal is defined as a suitable halogen, Y¹ is defined as (CH₂)ₘ, and m is defined as in Formula (I), can be prepared according to Scheme 8,

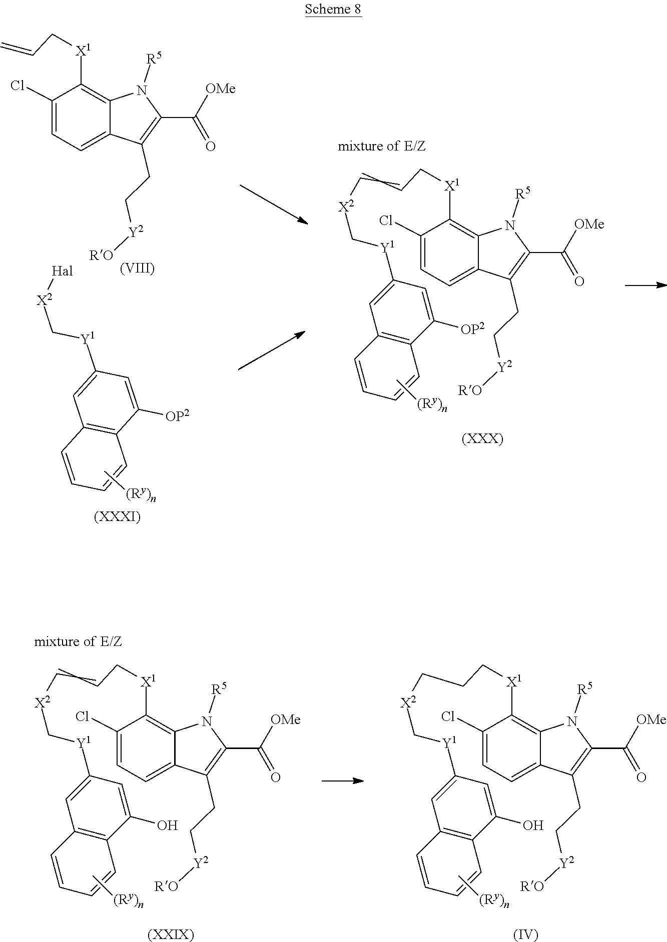

by reacting an intermediate of Formula (XXIX), with a suitable hydrogenating agent such as, for example, hydrogen gas, in the presence of a suitable catalyst such as, for example, Pd/C 10%, in a suitable solvent such as, for example, MeOH, at a suitable temperature such as, for example, room temperature.
Intermediates of Formula (XXIX), can be prepared by reacting an intermediate of Formula (XXX), wherein P² is a suitable protecting group such as, for example, TBDPS, with a suitable deprotecting agent such as, for example, TBAF, in a suitable solvent such as, for example, THF, at a suitable temperature such as, for example, room temperature.
Intermediates of Formula (XXX) can be prepared by reacting an intermediate of Formula (VIII) with an intermediate of Formula (XXXI), in the presence of a suitable base such as, for example, DIPEA, in the presence of a suitable catalyst such as, for example, Pd(tBu₃P)₂, in a suitable solvent such as, for example, DMF, at a suitable temperature such as, for example, 120° C.

Intermediates of Formula (XXXI), wherein X², and R^{y} and n are defined as in Formula (I), Y² is CH₂, P² is a suitable protecting group such as, for example, TBDPS, and Hal is defined as a suitable halogen such as, for example, bromide, can be prepared according to Scheme 9,

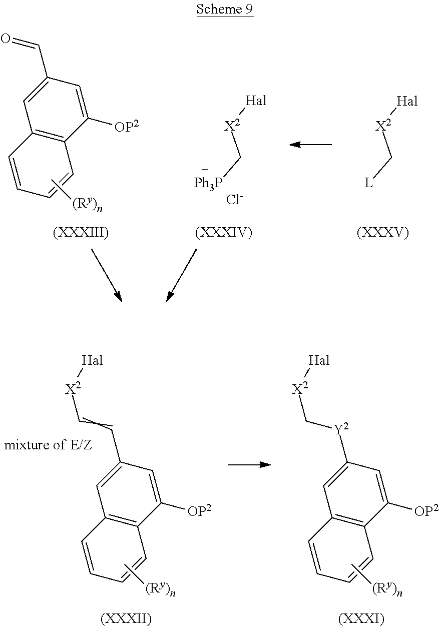

by reacting an intermediate of Formula (XXXII) with a suitable hydrogenating agent such as, for example, hydrogen, in the presence of a suitable catalyst such as, for example, PtO₂, in a suitable solvent such as, for example, EtOAc, at a suitable temperature such as, for example, room temperature.
Intermediates of Formula (XXXII) can be prepared by reacting an intermediate of Formula (XXXIII) with an intermediate of Formula (XXXIV) in the presence of a suitable base such as, for example, NaH, in a suitable solvent such as, for example, THF, at a suitable temperature such as, for example, −30° C. or 0° C.
Intermediates of Formula (XXXIV) can be prepared by reacting a suitable haloheterocycle of Formula (XXXV), wherein L is a suitable leaving group, such as, for example, 3-bromo-5-(chloromethyl)-1-methyl-1H-pyrazole (CAS [2109428-60-4]), with a suitable phosphine such as, for example, PPh₃, in a suitable solvent such as, for example, acetonitrile (ACN), at a suitable temperature such as, for example, 85° C.

Intermediates of Formula (XXXIII), wherein R^{y} and n are defined as in Formula (I), and P² is a suitable protecting group such as, for example, TBDPS, can be prepared according to Scheme 10

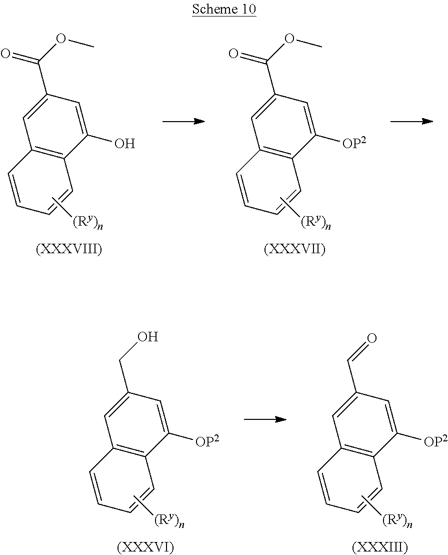

by reacting an intermediate of Formula (XXXVI) with a suitable oxidizing agent such as, for example, MnO₂, in a suitable solvent such as, for example, acetonitrile, at a suitable temperature such as, for example, 60° C.
Intermediates of Formula (XXXVI) can be prepared by reacting an intermediate of Formula (XXXVII) with a suitable reducing agent such as, for example, LiAlH₄, in a suitable solvent such as, for example, THF, at a suitable temperature such as, for example, 0° C.
Intermediates of Formula (XXXVII) can be prepared by reacting an intermediate of Formula (XXXVIII) with a suitable protecting reagent such as, for example, tert-butyl(chloro)diphenylsilane (TBDPSCl) or 4-methoxybenzyl chloride (PMBCl), in the presence of a suitable base such as, for example, imidazole or NaH, in a suitable solvent such as, for example, DMF, at a suitable temperature such as, for example, room temperature.
Intermediates of Formula (XXXVIII) are commercially available or can be prepared according to procedures described in literature.

Intermediates of Formula (XXXV), wherein X² is defined as in Formula (I), L is a suitable leaving group such as, for example, chloride, and Hal is defined as a suitable halogen such as, for example, bromide, can be prepared according to Scheme 11,

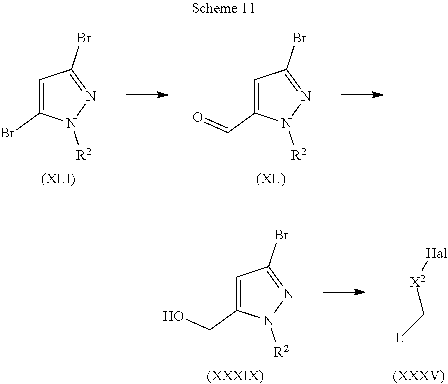

by reacting an intermediate of Formula (XXXIX) with a suitable activating agent such as, for example, thionyl chloride, in a suitable solvent such as, for example, DCM, at a suitable temperature such as, for example, room temperature.
Intermediates of Formula (XXXIX) can be prepared by reacting an intermediate of Formula (XL) with a suitable reducing agent such as, for example, NaBH₄, in a suitable solvent such as, for example, MeOH, at a suitable temperature such as, for example, 15° C.
Intermediates of Formula (XL) can be prepared by reacting an intermediate of Formula (XLI) with a suitable acylating agent such as, for example, DMF, in the presence of a suitable base such as, for example, BuLi, in a suitable solvent such as, for example, THF, at a suitable temperature such as, for example, −78° C.
Intermediates of Formula (XLI) are commercially available or can be prepared according to procedures described in literature.

It will be appreciated that where appropriate functional groups exist, compounds of various formulae or any intermediates used in their preparation may be further derivatized by one or more standard synthetic methods employing condensation, substitution, oxidation, reduction, or cleavage reactions. Particular substitution approaches include conventional alkylation, arylation, heteroarylation, acylation, sulfonylation, halogenation, nitration, formylation and coupling procedures.

The compounds of Formula (I) may be synthesized in the form of racemic mixtures of enantiomers which can be separated from one another following art-known resolution procedures. The racemic compounds of Formula (I) containing a basic nitrogen atom may be converted into the corresponding diastereomeric salt forms by reaction with a suitable chiral acid. Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

In the preparation of compounds of the present invention, protection of remote functionality (e.g., primary or secondary amine) of intermediates may be necessary. The need for such protection will vary depending on the nature of the remote functionality and the conditions of the preparation methods. The need for such protection is readily determined by one skilled in the art. For a general description of protecting groups and their use, see T. W. Greene and P. G. M. Wuts, Protective Groups in Organic Synthesis, 4th ed., Wiley, Hoboken, N.J., 2007.

### Pharmacology of Compounds

It has been found that the compounds of the present invention inhibit one of more MCL-1 activities, such as MCL-1 antiapoptotic activity.

An MCL-1 inhibitor is a compound that blocks one or more MCL-1 functions, such as the ability to bind and repress proapoptotic effectors Bak and Bax or BH3 only sensitizers such as Bim, Noxa or Puma.

The compounds of the present invention can inhibit the MCL-1 pro-survival functions. Therefore, the compounds of the present invention may be useful in treating and/or preventing, in particular treating, diseases that are susceptible to the effects of the immune system such as cancer.

In another embodiment of the present invention, the compounds of the present invention exhibit anti-tumoral properties, for example, through immune modulation.

In an embodiment, the present invention is directed to methods for treating and/or preventing a cancer, wherein the cancer is selected from those described herein, comprising administering to a subject in need thereof (preferably a human), a therapeutically effective amount of a compound of Formula (I), or pharmaceutically acceptable salt, or a solvate thereof.

In an embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of acute lymphoblastic leukemia (ALL), acute myeloid leukemia (AML), B cells acute lymphoblastic leukemia, B-cell chronic lymphocytic leukemia (CLL), bladder cancer, breast cancer, chronic lymphocytic leukemia, chronic myeloid leukemia, colon adenocarcinoma, diffuse large B cell lymphoma, esophageal cancer, follicular lymphoma, gastric cancer, head and neck cancer (including, but not limited to head and neck squamous cell carcinoma), hematopoietic cancer, hepatocellular carcinoma, Hodgkin lymphoma, liver cancer, lung cancer (including but not limited to lung adenocarcinoma), lymphoma, medulloblastoma, melanoma, monoclonal gammopathy of undetermined significance, multiple myeloma, myelodysplastic syndromes, myelofibrosis, myeloproliferative neoplasms, ovarian cancer, ovarian clear cell carcinoma, ovarian serous cystadenoma, pancreatic cancer, polycythemia vera, prostate cancer, rectum adenocarcinoma, renal cell carcinoma, smoldering multiple myeloma, T cell acute lymphoblastic leukemia, T cell lymphoma, and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is preferably selected from the group consisting of acute lymphoblastic leukemia (ALL), acute myeloid leukemia (AML), B cells acute lymphoblastic leukemia, B-cell chronic lymphocytic leukemia (CLL), breast cancer, chronic lymphocytic leukemia, chronic myeloid leukemia, diffuse large B cell lymphoma, follicular lymphoma, hematopoietic cancer, Hodgkin lymphoma, lung cancer (including, but not limited to lung adenocarcinoma) lymphoma, monoclonal gammopathy of undetermined significance, multiple myeloma, myelodysplastic syndromes, myelofibrosis, myeloproliferative neoplasms, smoldering multiple myeloma, T cell acute lymphoblastic leukemia, T cell lymphoma and Waldenstroms macroglobulinemia.

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of adenocarcinoma, benign monoclonal gammopathy, biliary cancer (including, but not limited to, cholangiocarcinoma), bladder cancer, breast cancer (including, but not limited to, adenocarcinoma of the breast, papillary carcinoma of the breast, mammary cancer, medullary carcinoma of the breast), brain cancer (including, but not limited to, meningioma), glioma (including, but not limited to, astrocytoma, oligodendroglioma; medulloblastoma), bronchus cancer, cervical cancer (including, but not limited to, cervical adenocarcinoma), chordoma, choriocarcinoma, colorectal cancer (including, but not limited to, colon cancer, rectal cancer, colorectal adenocarcinoma), epithelial carcinoma, endothelial sarcoma (including, but not limited to, Kaposi's sarcoma, multiple idiopathic hemorrhagic sarcoma), endometrial cancer (including, but not limited to, uterine cancer, uterine sarcoma), esophageal cancer (including, but not limited to, adenocarcinoma of the esophagus, Barrett's adenocarinoma), Ewing sarcoma, gastric cancer (including, but not limited to, stomach adenocarcinoma), gastrointestinal stromal tumor (GIST), head and neck cancer (including, but not limited to, head and neck squamous cell carcinoma), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, kidney cancer (including, but not limited to, nephroblastoma a.k.a. Wilms' tumor, renal cell carcinoma), liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES), ovarian cancer (including, but not limited to, cystadenocarcinoma, ovarian embryonal carcinoma, ovarian adenocarcinoma), pancreatic cancer (including, but not limited to, pancreatic andenocarcinoma, intraductal papillary mucinous neoplasm (IPMN), Islet cell tumors), prostate cancer (including, but not limited to, prostate adenocarcinoma), skin cancer (including, but not limited to, squamous cell carcinoma (SCC), keratoacanthoma (KA), melanoma, basal cell carcinoma (BCC)) and soft tissue sarcoma (e.g. malignant fibrous histiocytoma (MFH), liposarcoma, malignant peripheral nerve sheath tumor (MPNST), chondrosarcoma, fibrosarcoma, myxosarcoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of benign monoclonal gammopathy, breast cancer (including, but not limited to, adenocarcinoma of the breast, papillary carcinoma of the breast, mammary cancer, medullary carcinoma of the breast), hematopoietic cancers (including, but not limited to, leukemia such as acute lymphocytic leukemia (ALL) (including, but not limited to, B-cell ALL, T-cell ALL), acute myelocytic leukemia (AML) (e.g. B-cell AML, T-cell AML), chronic myelocytic leukemia (CML) (e.g. B-cell CML, T-cell CML), and chronic lymphocytic leukemia (CLL) (e.g. B-cell CLL, T-cell CLL), lymphoma such as Hodgkin lymphoma (HL) (including, but not limited to, B-cell HL, T-cell HL) and non-Hodgkin lymphoma (NHL) (e.g. B-cell NHL such as diffuse large cell lymphoma (DLCL) (e.g. diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (including, but not limited to, mucosa-associated lymphoid tissue (MALT) lymphomas, nodal marginal zone B-cell lymphoma. splenic marginal zone B-cell lymphoma), primary mediastinal B-cell lymphoma, Burkitt lymphoma, lymphoplasmacytic lymphoma (including, but not limited to, Waldenstrom's macro globulinemia), immunoblastic large cell lymphoma, hairy cell leukemia (HCL), precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g. cutaneous T-cell lymphoma (CTCL) (including, but not limited to, mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma, a mixture of one or more leukemia/lymphoma as described above, multiple myeloma (MM), heavy chain disease (including, but not limited to, alpha chain disease, gamma chain disease, mu chain disease), immunocytic amyloidosis, liver cancer (including, but not limited to, hepatocellular cancer (HCC), malignant hepatoma), lung cancer (including, but not limited to, bronchogenic carcinoma, non-small cell lung cancer (NSCLC), squamous lung cancer (SLC), adenocarcinoma of the lung, Lewis lung carcinoma, lung neuroendocrine tumors, typical carcinoid, atypical carcinoid, small cell lung cancer (SCLC), and large cell neuroendocrine carcinoma), myelodysplastic syndromes (MDS), myeloproliferative disorder (MPD), and prostate cancer (including, but not limited to, prostate adenocarcinoma).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is selected from the group consisting of prostate, lung, pancreatic, breast, ovarian, cervical, melanoma, B-cell chronic lymphocytic leukemia (CLL), acute myeloid leukemia (AML), and acute lymphoblastic leukemia (ALL).

In another embodiment, the present invention is directed to a method for treating and/or preventing cancer comprising administering to a subject in need thereof, preferably a human, a therapeutically effective amount of a compound of Formula (I), or a pharmaceutically acceptable salt, or a solvate thereof, wherein the cancer is multiple myeloma.

The compounds according to the present invention or pharmaceutical compositions comprising said compounds, may also have therapeutic applications in combination with immune modulatory agents, such as inhibitors of the PD1/PDL1 immune checkpoint axis, for example antibodies (or peptides) that bind to and/or inhibit the activity of PD-1 or the activity of PD-L1 and or CTLA-4 or engineered chimeric antigen receptor T cells (CART) targeting tumor associated antigens.

The compounds according to the present invention or pharmaceutical compositions comprising said compounds, may also be combined with radiotherapy or chemotherapeutic agents (including, but not limited to, anti-cancer agents) or any other pharmaceutical agent which is administered to a subject having cancer for the treatment of said subject's cancer or for the treatment or prevention of side effects associated with the treatment of said subject's cancer.

The compounds according to the present invention or pharmaceutical compositions comprising said compounds, may also be combined with other agents that stimulate or enhance the immune response, such as vaccines.

In an embodiment, the present invention is directed to methods for treating and/or preventing a cancer (wherein the cancer is selected from those described herein) comprising administering to a subject in need thereof (preferably a human), a therapeutically effective amount of co-therapy or combination therapy; wherein the co-therapy or combination therapy comprises a compound of Formula (I) of the present invention and one or more anti-cancer agent(s) selected from the group consisting of (a) immune modulatory agent (such as inhibitors of the PD1/PDL1 immune checkpoint axis, for example antibodies (or peptides) that bind to and/or inhibit the activity of PD-1 or the activity of PD-L1 and or CTLA-4); (b) engineered chimeric antigen receptor T cells (CART) targeting tumor associated antigens; (c) radiotherapy; (d) chemotherapy; and (e) agents that stimulate or enhance the immune response, such as vaccines.

The present invention is directed to compounds of Formula (I) and pharmaceutically acceptable salts, and solvates thereof, for use as a medicament.

The present invention is directed to compounds of Formula (I) and pharmaceutically acceptable salts, and solvates thereof, for use in the inhibition of MCL-1 activity.

As used herein, unless otherwise noted, the term “anti-cancer agents” shall encompass “anti-tumor cell growth agents” and “anti-neoplastic agents”.

The present invention is directed to compounds of Formula (I) and pharmaceutically acceptable salts, and solvates thereof, for use in treating and/or preventing diseases (preferably cancers) mentioned above.

The present invention is directed to compounds of Formula (I) and pharmaceutically acceptable salts, and solvates thereof, for treating and/or preventing diseases (preferably cancers) mentioned above.

The present invention is directed to compounds of Formula (I) and pharmaceutically acceptable salts, and solvates thereof, for treating and/or preventing, in particular for treating, a disease, preferably a cancer, as described herein (for example, multiple myeloma).

The present invention is directed to compounds of Formula (I) and pharmaceutically acceptable salts, and solvates thereof, for use in treating and/or preventing, in particular for treating, a disease, preferably a cancer, as described herein (for example, multiple myeloma).

The present invention is directed to compounds of Formula (I) and pharmaceutically acceptable salts, and solvates thereof, for treating and/or preventing, in particular for treating, MCL-1 mediated diseases or conditions, preferably cancer, more preferably a cancer as herein described (for example, multiple myeloma).

The present invention is directed to compounds of Formula (I) and pharmaceutically acceptable salts, and solvates thereof, for use in treating and/or preventing, in particular for use in treating, MCL-1 mediated diseases or conditions, preferably cancer, more preferably a cancer as herein described (for example, multiple myeloma).

The present invention relates to compounds of Formula (I) and pharmaceutically acceptable salts, and solvates thereof, for the manufacture of a medicament.

The present invention relates to compounds of Formula (I) and pharmaceutically acceptable salts, and solvates thereof, for the manufacture of a medicament for the inhibition of MCL-1.

The present invention relates to compounds of Formula (I) and pharmaceutically acceptable salts, and solvates thereof, for the manufacture of a medicament for treating and/or preventing, in particular for treating, a cancer, preferably a cancer as herein described. More particularly, the cancer is a cancer which responds to inhibition of MCL-1 (for example, multiple myeloma).

The present invention is directed to compounds of Formula (I) and pharmaceutically acceptable salts, and solvates thereof, for the manufacture of a medicament for treating and/or preventing, in particular for treating, any one of the disease conditions mentioned hereinbefore.

The present invention is directed to compounds of Formula (I) and pharmaceutically acceptable salts, and solvates thereof, for the manufacture of a medicament for treating and/or preventing any one of the disease conditions mentioned hereinbefore.

The compounds of Formula (I) and pharmaceutically acceptable salts, and solvates thereof, can be administered to subjects, preferably humans, for treating and/or preventing of any one of the diseases mentioned hereinbefore.

In view of the utility of the compounds of Formula (I) and pharmaceutically acceptable salts, and solvates thereof, there is provided a method of treating subjects, preferably mammals such as humans, suffering from any of the diseases mentioned hereinbefore; or a method of slowing the progression of any of the diseases mentioned hereinbefore in subject, humans; or a method of preventing subjects, preferably mammals such as humans, from suffering from any one of the diseases mentioned hereinbefore.

Said methods comprise the administration, i.e. the systemic or topical administration, preferably oral or intravenous administration, more preferably oral administration, of an effective amount of a compound of Formula (I) or a pharmaceutically acceptable salt, or a solvate thereof, to subjects such as humans.

One skilled in the art will recognize that a therapeutically effective amount of the compounds of the present invention is the amount sufficient to have therapeutic activity and that this amount varies inter alias, depending on the type of disease, the concentration of the compound in the therapeutic formulation, and the condition of the patient. In an embodiment, a therapeutically effective daily amount may be from about 0.005 mg/kg to 100 mg/kg.

The amount of a compound according to the present invention, also referred to herein as the active ingredient, which is required to achieve a therapeutic effect may vary on case-by-case basis, for example with the specific compound, the route of administration, the age and condition of the recipient, and the particular disorder or disease being treated. The methods of the present invention may also include administering the active ingredient on a regimen of between one and four intakes per day. In these methods of the present invention, the compounds according to the invention are preferably formulated prior to administration.

The present invention also provides compositions for treating and/or preventing the disorders (preferably a cancer as described herein) referred to herein. Said compositions comprise a therapeutically effective amount of a compound of Formula (I), or a pharmaceutically acceptable salt, or a solvate thereof, and a pharmaceutically acceptable carrier or diluent.

While it is possible for the active ingredient (e.g. a compound of the present invention) to be administered alone, it is preferable to administer it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of the present invention may be prepared by any methods well known in the art of pharmacy, for example, using methods such as those described in, for example, Gennaro et al. Remington's Pharmaceutical Sciences (18^{th} ed., Mack Publishing Company, 1990, see especially Part 8: Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, in an embodiment, the present invention is directed to a product comprising, as a first active ingredient a compound according to the invention and as further, as an additional active ingredient one or more anti-cancer agent(s), as a combined preparation for simultaneous, separate or sequential use in the treatment of patients suffering from cancer.

The one or more other anti-cancer agents and the compound according to the present invention may be administered simultaneously (e.g. in separate or unitary compositions) or sequentially, in either order. In an embodiment, the two or more compounds are administered within a period and/or in an amount and/or a manner that is sufficient to ensure that an advantageous or synergistic effect is achieved. It will be appreciated that the preferred method and order of administration and the respective dosage amounts and regimes for each component of the combination will depend on the particular other anti-cancer agent and the compound of the present invention being administered, their route of administration, the particular condition, in particular tumor, being treated and the particular host being treated.

The following examples further illustrate the present invention.

### EXAMPLES

Several methods for preparing the Compounds of this invention are illustrated in the following examples. Unless otherwise noted, all starting materials were obtained from commercial suppliers and used without further purification, or alternatively can be synthesized by a skilled person by using well-known methods.

| Abbreviation | Meaning |
|---|---|
| ACN | acetonitrile |
| BuLi | butyl lithium |
| DCM | dichloromethane |
| DIPE | diisopropyl ether |
| DIPEA | N,N-diisopropylethylamine |
| DMF | N,N-dimethylformamide |
| DTBAD | Di-tert-butyl Azodicarboxylate |
| Et₃N or TEA | trietylamine |
| EtOAc | ethyl acetate |
| EtOH | ethanol |
| h | hour(s) |
| HPLC | high performance liquid chromatography |
| iPrOH | 2-propanol |
| iPrNH₂ | isopropylamine |
| MeOH | methanol |
| min | minute(s) |
| MsCl | methanesulfonyl chloride |
| BuLi | butyllithium |
| NBS | N-Bromosuccinimide |
| Pd/C | palladium on carbon |
| PPh₃ | triphenylphosphine |
| Pd(PtBu₃)₂ | bis(tri-tert-butylphosphine)palladium(0) |
| P(nBu)₃ | tri-n-butylphosphine |
| Pd(AmPhos)₂Cl₂ | bis(di-tert-butyl(4- |
| | dimethylaminophenyl)phosphine)dichloropalladium(II) |
| PTSA | p-toluenesulfonic acid |
| RP | reversed phase |
| SFC | supercritical fluid chromatography |
| TBAF | tetrabutylammonium fluoride |
| THF | tetrahydrofuran |
| THP | tetrahydropyran |
| TBDPSCl | tert-butyl(chloro)diphenylsilane |

As understood by a person skilled in the art, Compounds synthesized using the protocols as indicated may contain residual solvent or minor impurities.

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

### Preparation of Intermediates

For intermediates that were used in a next reaction step as a crude or as a partially purified intermediate, in some cases no mol amounts are mentioned for such intermediate in the next reaction step or alternatively estimated mol amounts or theoretical mol amounts for such intermediate in the next reaction step are indicated in the reaction protocols described below.

### Intermediate 1a and Intermediate 1b

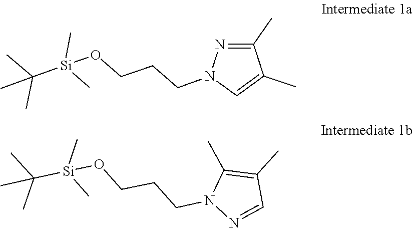

NaH (60% in mineral oil, 8.73 g, 1.05 eq.) was added portionwise to a stirred solution of 3,4-dimethyl-1H-pyrazole (CAS [2820-37-3]) (20 g, 208 mmol) and (3-bromopropoxy)-tert-butyldimethylsilane (CAS [89031-84-5]) (55.32 g, 1.05 eq.) in DMF (400 mL) at 0° C. The reaction mixture was allowed to warm up to room temperature and was stirred for 30 min. The reaction was quenched by addition of saturated aqueous NH₄Cl (200 mL) and water (200 mL). The mixture was extracted with EtOAc (3×200 mL). The combined organic layer was washed with water (400 mL) and brine (300 mL), dried over MgSO₄, filtered, and concentrated under reduced pressure to afford a mixture of Intermediate 1a and Intermediate 1b (63.2 g, yield: 56%) as a yellow liquid.

### Intermediate 2

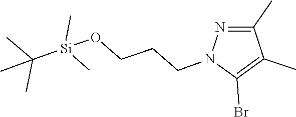 [SMILES: Cc1nn(CCCO[Si](C)(C)C(C)(C)C)c(Br)c1C]

NBS (44 g, 247.18 mmol, 2.1 eq.) was added to a solution of the mixture of Intermediate 1a and Intermediate 1b (63.2 g, 117.7 mmol) in DCM (600 mL) at 0° C. under nitrogen atmosphere. The reaction mixture was allowed to warm up to room temperature and was stirred for 1 h. The reaction mixture was diluted carefully with saturated aqueous Na₂SO₃ (200 mL). The yellow solution was stirred at room temperature for 10 min. The organic layer was separated, dried over MgSO₄, filtered, and concentrated under reduced pressure. The residue was purified by flash column chromatography on silica gel (330 g, gradient: petroleum ether/EtOAc 100/0 to 90/10) to give a yellow liquid that was further purified by preparative HPLC (Stationary phase: RP Xtimate Prep C18 OBD—5 μm, 40×150 mm, Mobile phase: water (10 mM NH₄HCO₃)/CH₃CN 10/90 to 3/97). The obtained product was diluted with water (70 mL) and EtOAc (150 mL). The organic layer was separated, dried over MgSO₄, filtered, and concentrated under reduced pressure to give Intermediate 2 (24 g, yield: 59%) as a yellow liquid.

### Intermediate 3

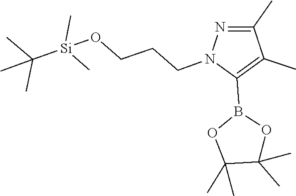 [SMILES: Cc1nn(CCCO[Si](C)(C)C(C)(C)C)c(B2OC(C)(C)C(C)(C)O2)c1C]

BuLi (2.5 M in hexane, 6.33 mL, 1.1 eq.) was added dropwise to a solution of Intermediate 2 (5 g, 14.39 mmol) in dry THF (75 mL) at −78° C., under nitrogen atmosphere. The reaction mixture was stirred at −78° C. for 45 min, before the dropwise addition of 2-isopropoxy-4,4,5,5-tetramethyl-1,3,2-dioxaborolane [61676-62-8] (3.52 mL, 1.2 eq.). The reaction mixture was allowed to warm up to room temperature and was stirred for 16 h. The reaction was quenched by addition of saturated aqueous NH₄Cl (25 mL). The reaction mixture was diluted with water (50 mL) and EtOAc (100 mL) and the layers were separated. The aqueous layer was back-extracted with EtOAc (75 mL). The combined organic layer was dried over MgSO₄, filtered, and evaporated. The residue was purified by flash column chromatography on silica gel (120 g, gradient: heptane/EtOAc 100/0 to 80/20) to afford Intermediate 3 (4.19 g, yield: 74%) as a colourless oil.

### Intermediate 4

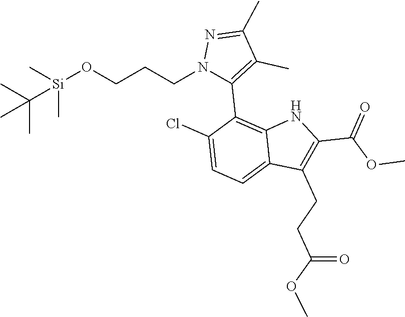 [SMILES: COC(=O)CCc1c(C(=O)OC)[nH]c2c(-c3c(C)c(C)nn3CCCO[Si](C)(C)C(C)(C)C)c(Cl)ccc12]

A mixture of 1H-indole-3-propanoic acid, 7-bromo-6-chloro-2-(methoxycarbonyl)-, methyl ester (CAS [2143010-85-7]) (2 g, 5.34 mmol), Intermediate 3 (3.26 g, 1.55 eq.), and bis-di-tert-butyl(4-dimethylaminophenyl)phosphine)dichloropalladium (II) (CAS [887919-35-9]) (189 mg, 0.05 eq.) in 1,4-dioxane (26 mL) and water (5 mL) was degassed with nitrogen for a few min. K₂CO₃ (1.47 g, 2 eq.) was added and the reaction mixture was then stirred at 80° C. for 3 h. The reaction mixture was diluted with EtOAc (50 mL) and water (30 mL) and the layers were separated. The aqueous layer was back-extracted with EtOAc (30 mL). The combined organic layer was dried over MgSO₄, filtered, and evaporated to afford Intermediate 4 (1.8 g, yield: 60%), as a white solid, used without further purification.

### Intermediate 5

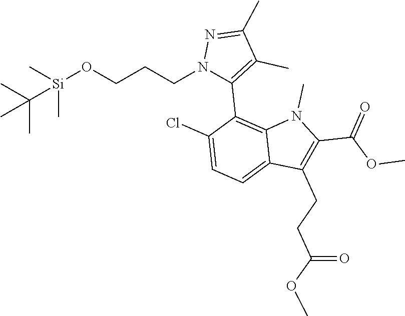 [SMILES: COC(=O)CCc1c(C(=O)OC)n(C)c2c(-c3c(C)c(C)nn3CCCO[Si](C)(C)C(C)(C)C)c(Cl)ccc12]

CH₃I (0.3 mL, 1.6 eq.) was added to a solution of Intermediate 4 (1.72 g, 3.06 mmol) and Cs₂CO₃ (1.5 g, 1.5 eq.) in DMF (12 mL). The reaction mixture was stirred at room temperature for 2 h. The mixture was diluted with EtOAc (75 mL) and water (50 mL). The organic layer was separated and washed with brine (3×30 mL). The aqueous layer was back-extracted with EtOAc (25 mL). The combined organic layer was dried over MgSO₄, filtered, and concentrated under reduced pressure. The residue was purified by flash column chromatography on silica gel (80 g, gradient: from heptane/EtOAc 100/0 to 70/30) to afford Intermediate 5 (1.67 g, yield: 95%) as a colourless paste.

### Intermediate 6

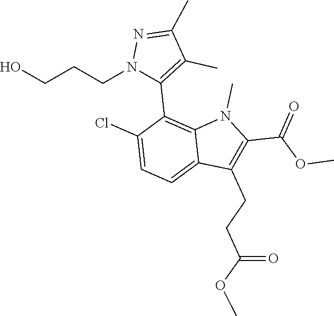 [SMILES: COC(=O)CCc1c(C(=O)OC)n(C)c2c(-c3c(C)c(C)nn3CCCO)c(Cl)ccc12]

TBAF (1 M in THF, 3.29 mL, 1.2 eq.) was added to a solution of Intermediate 5 (1.58 g, 2.74 mmol) in dry THF (30 mL) under nitrogen atmosphere. The reaction mixture was stirred at room temperature for 1 h. The solvent was evaporated and the residue was diluted with EtOAc (70 mL) and water (30 mL). The layers were separated and the aqueous layer was back-extracted with EtOAc (30 mL). The combined organic layer was dried over MgSO₄, filtered, and concentrated under reduced pressure. The residue was purified by flash column chromatography on silica gel (40 g, gradient: heptane/EtOAc 100/0 to 0/100) to afford Intermediate 6 (1.14 g, yield: 90%) as a colourless paste.

### Intermediate 7

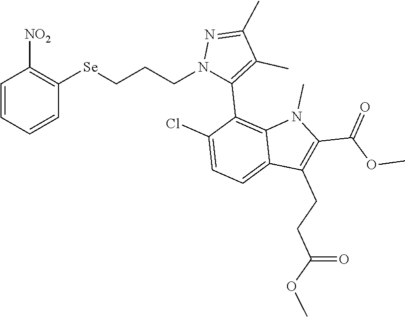 [SMILES: COC(=O)CCc1c(C(=O)OC)n(C)c2c(-c3c(C)c(C)nn3CCC[Se]c3ccccc3[N+](=O)[O-])c(Cl)ccc12]

Tri-n-butylphosphine (1.34 mL, 2.4 eq.) was added dropwise to a solution of Intermediate 6 (1.04 g, 2.25 mmol) and 2-nitrophenyl selenocyanate (CAS [51694-22-5]) (1.02 g, 2 eq.) in dry THF (20 mL) under nitrogen atmosphere at room temperature. The reaction mixture was stirred at room temperature for 30 min. The solvent was evaporated and the residue was purified by flash column chromatography on silica gel (40 g, gradient: heptane/EtOAc 100/0 to 30/70) to afford Intermediate 7 (1.35 g, yield: 93%) as a yellow paste.

### Intermediate 8

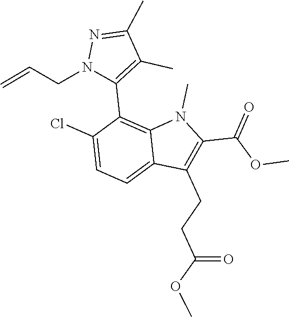 [SMILES: C=CCn1nc(C)c(C)c1-c1c(Cl)ccc2c(CCC(=O)OC)c(C(=O)OC)n(C)c12]

Hydrogen peroxide (1.15 mL, 5 eq.) was added dropwise to a solution of Intermediate 7 (1.49 g, 2.3 mmol) in THE (20 mL). The reaction mixture was stirred at room temperature for 3 h. The reaction mixture was diluted with EtOAc (30 mL) and water (20 mL). The layers were separated and the aqueous layer was back-extracted with EtOAc (20 mL).

The combined organic layer was dried over MgSO₄, filtered, and concentrated under reduced pressure. The residue was dissolved in DCM (25 mL) and filtered. The solid was discarded and the filtrate was evaporated. The residue was purified by flash column chromatography on silica gel (40 g, gradient: heptane/EtOAc 100/0 to 60/40) to afford Intermediate 8 (780 mg, yield: 76%) as a yellow paste.

### Intermediate 9

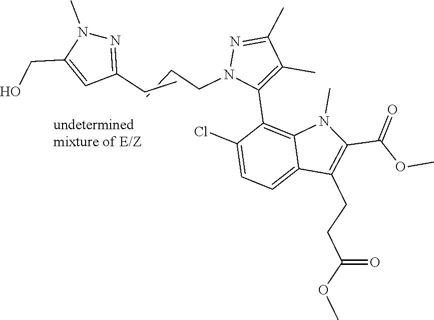

Intermediate 8 (680 mg, 1.53 mmol), (3-bromo-1-methyl-1H-pyrazol-5-yl)methanol (CAS [1784533-05-6]) (439 mg, 1.5 eq.) and bis(tri-tert-butylphosphine)palladium (CAS [53199-31-8]) (157 mg, 0.2 eq.) were placed in a sealed tube under nitrogen atmosphere. DMF (16 mL) was added and the solution was degassed by bubbling nitrogen for a few minutes, then DIPEA (0.78 mL, 3.0 eq.) was added and the reaction mixture was stirred at 120° C. for 2 h. The reaction mixture was diluted with EtOAc (50 mL) and water (30 mL). The organic layer was separated and washed with brine (2×25 mL). The aqueous layer was back-extracted with EtOAc (30 mL). The combined organic layer was dried over MgSO₄, filtered, and concentrated under reduced pressure. The residue was purified by flash column chromatography on silica gel (40 g, gradient: DCM/MeOH 100/0 to 95/5) to afford Intermediate 9 (628 mg, yield: 74%) as a foam.

### Intermediate 10

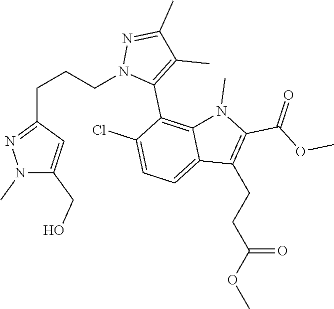

A solution of Intermediate 9 (725 mg, 1.31 mmol) in EtOAc (56 mL) was hydrogenated in the presence of Pd/C (10%) (139 mg, 0.1 eq.) at room temperature and atmospheric pressure for 6 h. The reaction mixture was filtered over Celite® and the filter pad was washed with EtOAc (20 mL). The filtrate was concentrated under reduced pressure and the residue was purified by flash column chromatography on silica gel (40 g, gradient: DCM/MeOH 100/0 to 95/5) to afford Intermediate 10 (523 mg, yield: 72%) as a foam.

### Intermediate 11

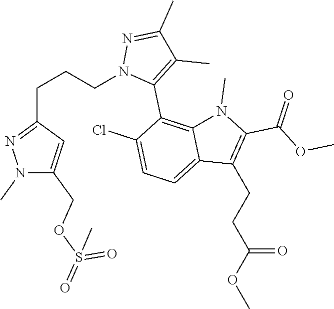

MsCl (182 μL, 2.5 eq.) was added dropwise to a stirred solution of Intermediate 10 (523 mg, 0.94 mmol) and Et₃N (392 μL, 3.0 eq.) in DCM (20 mL) at 0° C. under nitrogen atmosphere. The reaction mixture was then warmed up to room temperature and was stirred for 1 h. The reaction mixture was diluted with DCM (25 mL) and treated with saturated aqueous NaHCO₃ (20 mL). The organic layer was separated and the aqueous layer was back-extracted with DCM (25 mL). The combined organic layer was dried over MgSO₄, filtered, and concentrated under reduced pressure to afford Intermediate 11 (596 mg, assumed quantitative), used without further purification.

### Intermediate 12

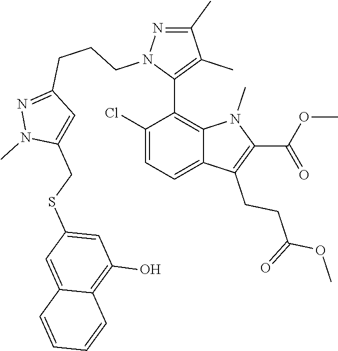

K₂CO₃ (195 mg, 1.5 eq.) was added to a stirred solution of ethanethioic acid, S-[4-(acetyloxy)-2-naphthalenyl] ester (CAS [2143010-96-0]) (318 mg, 1.3 eq.) in degassed MeOH (10 mL). After 5 min, Intermediate 11 (597 mg, 0.94 mmol) in THF (5 mL) was added dropwise. The reaction mixture was stirred at room temperature for 1 h. More K₂CO₃ (195 mg, 1.5 eq.) was added and the reaction mixture was stirred at room temperature for 30 min. The solvent was evaporated and EtOAc (50 mL) and water (30 mL) were added. The organic layer was separated and the aqueous layer was back-extracted with EtOAc (50 mL). The combined organic layer was dried over MgSO₄, filtered, and concentrated under reduced pressure. The residue was purified by flash column chromatography on silica gel (40 g, gradient: DCM/MeOH 100/0 to 97/3) to afford Intermediate 12 (360 mg, yield: 54%) as a foam.

### Intermediate 13

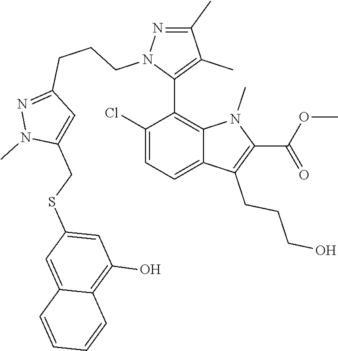

Borane dimethyl sulfide complex (2 M in THF, 997 μL, 5 eq.) was added to a stirred solution of Intermediate 12 (285 mg, 0.4 mmol) in THF (6 mL) under nitrogen atmosphere. The reaction mixture was stirred at 50° C. for 5 h. The reaction mixture was cooled down to room temperature and treated with MeOH (10 mL) added dropwise, followed by aqueous HCl (1 M, 3 mL). This mixture was stirred at room temperature for 16 h before it was concentrated. The residue was taken up in DCM (40 mL) and water (5 mL). The organic layer was separated and the aqueous layer was back-extracted with DCM (2×10 mL). The combined organic layer was dried over MgSO₄, filtered, and concentrated under reduced pressure. The residue was purified by flash column chromatography on silica gel (40 g, gradient: DCM/MeOH 100/0 to 97/3) to afford Intermediate 13 (178 mg, yield: 65%) as a white foam.

### Intermediate 14 and Intermediate 15

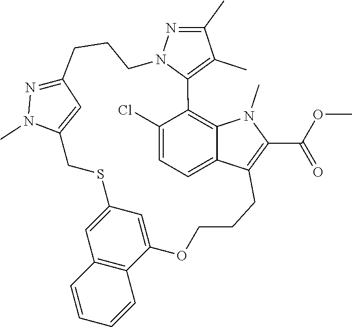

Intermediate 14: Rₐ or Sₐ; one atropisomer but absolute stereochemistry undetermined
Intermediate 15: Sₐ or Rₐ; one atropisomer but absolute stereochemistry undetermined

A solution of DTBAD (302 mg, 4 eq.) and Intermediate 13 (225 mg, 0.328 mmol) in toluene (6 mL) and THE (1.2 mL) was added with a syringe pump (0.1 mL/min) to a stirred solution of PPh₃ (344 mg, 4 eq.) in toluene (6 mL) at 70° C. Once the addition was complete, the reaction mixture was allowed to cool down to room temperature and the solvents were evaporated. The residue was purified by flash column chromatography on silica gel (40 g, gradient: DCM/MeOH 100/0 to 96/4). The white foam that was obtained was further separated into its atropisomers by preparative SFC (Stationary phase: Chiralpak Diacel ID 20×250 mm, Mobile phase: CO₂, EtOH+0.4% iPrNH₂) to afford Intermediate 14 (62 mg, yield: 28%) and Intermediate 15 (68 mg, yield: 31%).

### Intermediate 16

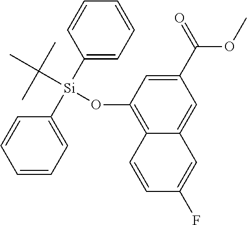 [SMILES: COC(=O)c1cc(O[Si](c2ccccc2)(c2ccccc2)C(C)(C)C)c2ccc(F)cc2c1]

TBDPSCl (14.66 g, 1.5 eq.) was added to a solution of methyl 7-fluoro-4-hydroxy-2-naphthoate (CAS [2092726-85-5], 8 g, 35.555 mmol) and imidazole (7.26, 3 eq.) in DCM (500 mL), cooled to 0° C. under nitrogen atmosphere. Once the addition was complete, the reaction was stirred at room temperature overnight. The reaction was quenched by addition of water (100 mL). The mixture was extracted with EtOAc (3×200 mL). The combined organic layer was dried over Na₂SO₄, filtered, and concentrated to afford a yellow oil. This oil was purified by flash column chromatography on silica gel (petroleum ether:EtOAc—1:0 to 1:1) to afford Intermediate 16 (14 g, yield: 86%) as a yellow oils.

### Intermediate 17

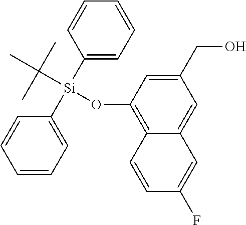 [SMILES: CC(C)(C)[Si](Oc1cc(CO)cc2cc(F)ccc12)(c1ccccc1)c1ccccc1]

LiAlH₄ (1.39 g, 1.2 eq.) was added slowly to a solution of Intermediate 16 (14 g, 30.528 mmol) in THF (200 mL), cooled to 0° C. under nitrogen atmosphere. Once the addition was complete the reaction mixture was stirred at 0° C. for 2 h. The reaction was quenched by slow addition of water (2 mL) followed by a 10% aqueous NaOH solution (2 mL) at 0° C. The heterogeneous mixture was filtered, and the filter cake was washed with DCM (200 mL). The filtrate was evaporated and the residue was purified by flash column chromatography on silica gel (petroleum ether:EtOAc—1:0 to 1:1) to give Intermediate 17 (12 g, yield: 90%) as a yellow solid.

### Intermediate 18

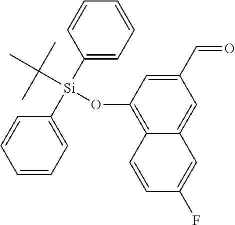 [SMILES: CC(C)(C)[Si](Oc1cc(C=O)cc2cc(F)ccc12)(c1ccccc1)c1ccccc1]

MnO₂ (29.074 g, 12 eq.) was added to a solution of Intermediate 17 (12 g, 27.869 mmol) in DCM (200 mL) at room temperature. The resulting solution was stirred at room temperature overnight. The reaction mixture was filtered and the filtrate was concentrated. The residue was purified by flash column chromatography over silica gel (eluent: petroleum ether/EtOAc, 100/0 to 50/50) to afford Intermediate 18 (12 g, yield: 99%) as a yellow oil.

### Intermediate 19

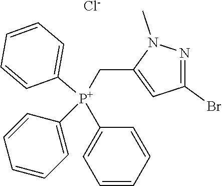 [SMILES: [Cl-] ; Cn1nc(Br)cc1C[P+](c1ccccc1)(c1ccccc1)c1ccccc1]

PPh₃ (19.53 g, 1.2 eq.) was added to a solution of 3-bromo-5-(chloromethyl)-1-methyl-1H-pyrazole (CAS [2109428-60-4], 13 g, 62 mmol) in ACN (150 mL) and the reaction mixture was stirred at 85° C. for 16 h. The solvent was evaporated. The residue was added to petroleum ether (100 mL) and this mixture was stirred at room temperature for 1 h. The solid was filtered and dried under vacuum to give Intermediate 19 (25 g, yield: 84%) as a white solid.

### Intermediate 20

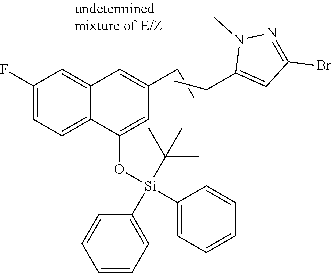

NaH (60% in mineral oil, 975 mg, 1.3 eq.) was added to a solution of Intermediate 19 (9 g, 18.76 mmol) in THE (100 mL) at 0° C. and the reaction mixture was stirred at 0° C. for 1 h. Intermediate 18 (9.79 g, 1.1 eq.) was then added at −30° C. and the reaction mixture was stirred at −30° C. for 2 h. The reaction was quenched by addition of aqueous NH₄Cl (50 mL). The mixture was extracted with EtOAc (100 mL×3). The combined organic layer was dried with Na₂SO₄ and the solvent was evaporated to give the crude product as a yellow oil. This oil was purified by column chromatography on silica gel (eluent: petroleum ether/EtOAc 100/0 to 85/15) to give Intermediate 20 (9 g, yield: 81%) as a white solid.

### Intermediate 21

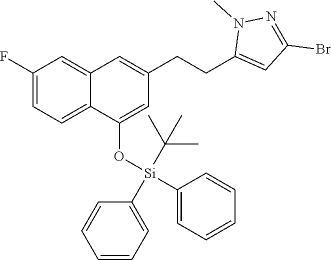 [SMILES: Cn1nc(Br)cc1CCc1cc(O[Si](c2ccccc2)(c2ccccc2)C(C)(C)C)c2ccc(F)cc2c1]

PtO₂ (1.04 g, 0.3 eq.) was added to a solution of Intermediate 20 (9 g, 15.25 mmol) in EtOAc (100 mL) under hydrogen atmosphere. The reaction mixture was stirred at room temperature for 6 h. The reaction mixture was filtered and the filtrate was evaporated. The residue was purified by column chromatography on silica gel (eluent: petroleum ether/EtOAc 100/0 to 85/15) to give Intermediate 21 (7.4 g, yield: 83%) as a white solid.

### Intermediate 22

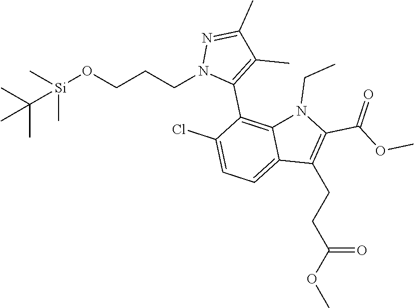 [SMILES: CCn1c(C(=O)OC)c(CCC(=O)OC)c2ccc(Cl)c(-c3c(C)c(C)nn3CCCO[Si](C)(C)C(C)(C)C)c21]

Iodoethane (1.45 g, 1.6 eq.) was added to a suspension of Intermediate 4 (3.27 g, 5.82 mmol) and Cs₂CO₃ (2.84 g, 1.5 eq.) in DMF (20 mL), stirring at room temperature. The reaction mixture was stirred at room temperature for 16 h. The mixture was diluted with EtOAc (100 mL) and water (80 mL). The aqueous layer was separated and the organic one was washed with brine (3×30 mL). The combined aqueous layer was back-extracted with EtOAc (50 mL). The combined organic layer was dried over MgSO₄, filtered, and evaporated. The residue was purified by flash column chromatography on silica gel (80 g, gradient: heptane/EtOAc 10/0 to 7/3) to afford Intermediate 22 (2.6 g, yield: 76%) as a yellowish paste.

### Intermediate 23

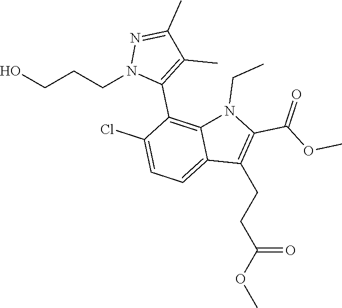 [SMILES: CCn1c(C(=O)OC)c(CCC(=O)OC)c2ccc(Cl)c(-c3c(C)c(C)nn3CCCO)c21]

TBAF (5.5 mL, 1 M in THF, 1.25 eq.) was added to a solution of Intermediate 22 (2.6 g, 4.405 mmol) in anhydrous THF (40 mL) stirring at room temperature under nitrogen atmosphere. The reaction mixture was stirred at room temperature for 2 h. The mixture was concentrated under reduced pressure and the residue was dissolved in EtOAc (120 mL) and washed with water (50 mL) and brine (50 mL). The combined aqueous layer was back-extracted with EtOAc (50 mL). The combined organic layer was dried over MgsO₄, filtered, and evaporated. The residue was purified by flash column chromatography on silica gel (80 g, gradient: heptane/EtOAc 100/0 to 0/100) to afford Intermediate 23 (1.39 g, yield: 80%) as a colorless paste.

### Intermediate 24

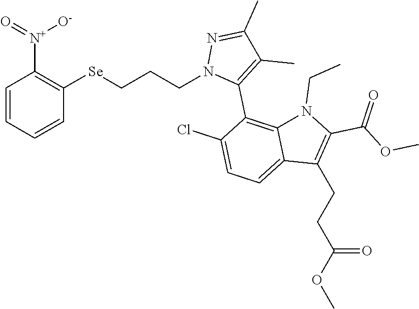 [SMILES: CCn1c(C(=O)OC)c(CCC(=O)OC)c2ccc(Cl)c(-c3c(C)c(C)nn3CCC[Se]c3ccccc3[N+](=O)[O-])c21]

nBu₃P (2.12 mL, 2.4 eq.) was added dropwise to a solution of Intermediate 23 (1.69 g, 3.55 mmol) and 2-nitrophenyl selenocyanate (1.61 g, 2 eq.) in anhydrous THF (30 mL) under nitrogen atmosphere at room temperature. The reaction mixture was stirred at room temperature for 30 min. Volatiles were removed in vacuo and the residue was purified by flash column chromatography on silica gel (80 g, heptane/EtOAc 100/0 to 50/50) to give Intermediate 24 (1.92 g, yield: 82%) as a yellow paste.

### Intermediate 25

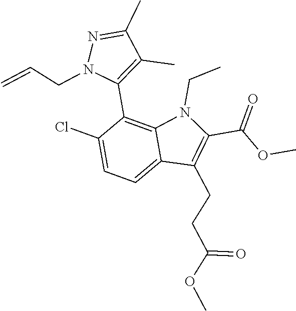 [SMILES: C=CCn1nc(C)c(C)c1-c1c(Cl)ccc2c(CCC(=O)OC)c(C(=O)OC)n(CC)c12]

Hydrogen peroxide (30%, 1.45 mL, 5 eq.) was added to a solution of Intermediate 24 (1.92 g, 2.91 mmol) in THF (25 mL). The reaction mixture was stirred at room temperature for 16 h. The reaction mixture was diluted with EtOAc (30 mL) and water (20 mL). The organic layer was separated and the aqueous one was back-extracted with EtOAc (20 mL). The combined organic layer was dried over MgSO₄, filtered, and evaporated. The residue was dissolved in DCM (˜30 mL) and filtered. The solid was discarded and the filtrate was evaporated. The residue was purified by flash column chromatography on silica gel (80 g, gradient: heptane/EtOAc 10/0 to 6/4) to afford Intermediate 25 (805 mg, yield: 60%) as a yellow paste.

### Intermediate 26

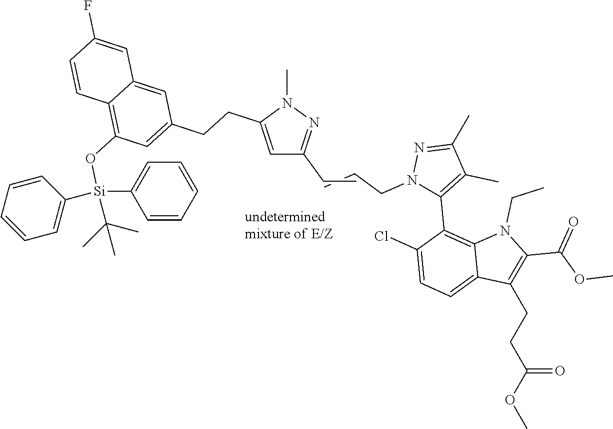

A vial was charged with Intermediate 25 (785 mg, 1.71 mmol), Intermediate 21 (1.51 g, 1.5 eq.) and Pd(PtBu₃)₂ (175 mg, 0.2 eq.) under nitrogen atmosphere. The vial was sealed and DMF (20 mL) was added via a syringe. The solution was degassed by bubbling nitrogen for a few min, then DIPEA (875 μL, 3 eq.) was added and the reaction mixture was heated to 120° C. for 3 h. The reaction mixture was cooled to room temperature and diluted with EtOAc (120 mL) and water (50 mL). The organic layer was separated and washed with brine (3×25 mL). The combined aqueous layer was back-extracted with EtOAc (50 mL). The combined organic layer was dried over MgSO₄, filtered, and evaporated. The residue was purified by flash chromatography on silica gel (120 g, gradient: DCM/MeOH 100/0 to 98/2) to give Intermediate 26 (1.24 g, yield: 75%) as a yellow paste.

### Intermediate 27

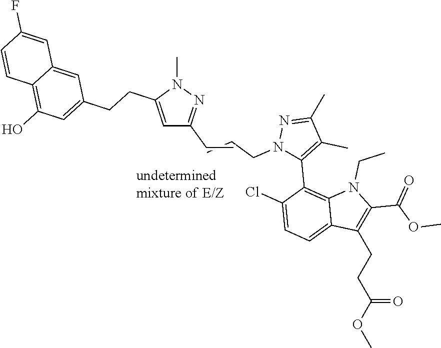

TBAF (1 M in THF, 2.6 mL) was added to a solution of Intermediate 26 (1.24 g, 1.285 mmol) in anhydrous THF (25 mL) stirring at room temperature under nitrogen atmosphere. The reaction mixture was stirred at room temperature for 3 h. The reaction mixture was concentrated under reduced pressure and the residue was dissolved in EtOAc (60 mL) and washed with water (25 mL) and brine (25 mL). The combined aqueous layer was back-extracted with EtOAc (25 mL). The combined organic layer was dried over MgsO₄, filtered, and evaporated. The residue was purified by flash column chromatography on silica gel (120 g, gradient: DCM/MeOH 100/0 to 97/3) to afford Intermediate 27 (460 mg, yield: 49%) as a white foam.

### Intermediate 28

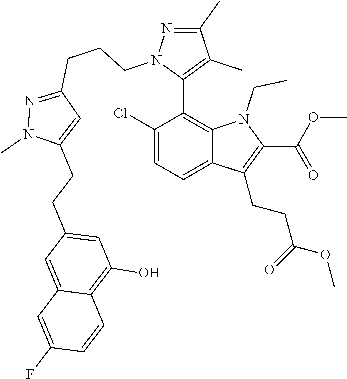

A solution of Intermediate 27 (460 mg, 0.633 mmol) in MeOH (20 mL) was hydrogenated in the presence of Pd/C (10%) (67 mg, 0.1 eq.) as catalyst while stirring for 2 h at room temperature. The reaction mixture was filtered over a pad of Dicalite®, washing the pad with MeOH. The filtrate was evaporated under reduced pressure to give Intermediate 28 (440 mg, yield: 95%) used in the next step without further purification.

### Intermediate 29

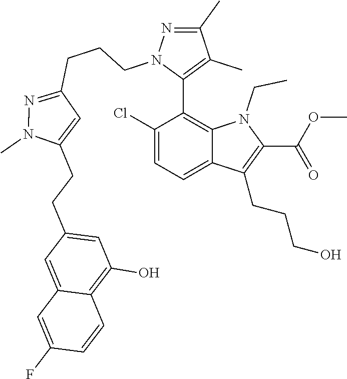

Borane dimethyl sulfide complex (2 M in THF, 1.51 mL, 5 eq.) was added to a solution of Intermediate 28 (440 mg, 0.604 mmol) in anhydrous THF (8 mL) under nitrogen atmosphere. The reaction mixture was stirred at 50° C. for 4 h. The reaction mixture was cooled to room temperature and treated with MeOH (10 mL, added dropwise initially) and HCl (1 N, 3 mL). This mixture was stirred at room temperature for 3 h. Volatiles were removed under reduced pressure and the residue was dissolved in DCM (40 mL) and water (20 mL). The organic layer was separated and the aqueous one was extracted with DCM (2×20 mL). The combined organic layer was dried over MgSO₄, filtered; and evaporated. The residue was purified by flash column chromatography on silica gel (40 g, gradient: DCM/MeOH 100/0 to 97/3) to give Intermediate 29 (365 mg, yield: 86%) as a foamy white solid.

### Intermediate 30 and Intermediate 31

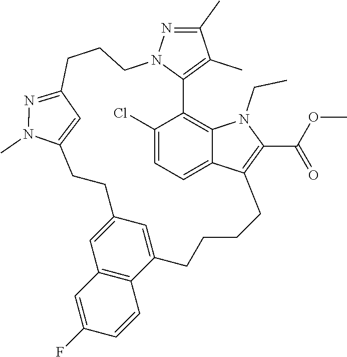

Intermediate 30: Rₐ or Sₐ; one atropisomer but absolute stereochemistry undetermined
Intermediate 31: Sₐ or Rₐ; one atropisomer but absolute stereochemistry undetermined

A solution of Intermediate 29 (365 mg, 0.521 mmol) and DTBAD (240 mg, 2 eq.) in toluene (10 mL) and THF (2 mL) was added with a syringe pump (0.1 mL/min) to a solution of PPh₃ (273 mg, 2 eq.) in toluene (10 mL), stirring at 70° C. Once the addition was complete, the reaction was allowed to cool to room temperature. Volatiles were removed under reduced pressure and the residue was purified by flash column chromatography on silica gel (80 g, gradient: DCM/MeOH 100/0 to 97/3) to obtain the desired racemic product as a foamy white solid. This racemic mixture was separated into its atropisomers by preparative SFC (Stationary phase: Chiralpak Diacel AD 20×250 mm, Mobile phase: CO₂, EtOH+0.4% iPrNH₂) to give Intermediate 30 (95 mg, yield: 27%) and Intermediate 31 (95 mg, yield: 27%).

### Intermediate 32

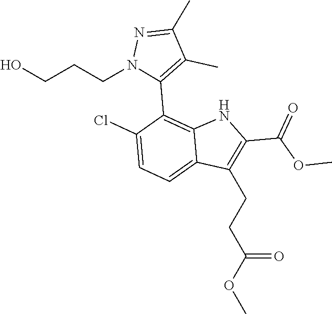 [SMILES: COC(=O)CCc1c(C(=O)OC)[nH]c2c(-c3c(C)c(C)nn3CCCO)c(Cl)ccc12]

TBAF (1 M in THF, 4.425 mL, 1.25 eq.) was added to a solution of Intermediate 4 (1.99 g, 3.54 mmol) in anhydrous THF (40 mL) under nitrogen atmosphere. The reaction mixture was stirred at room temperature for 3 h. The reaction mixture was concentrated under reduced pressure and the residue was dissolved in EtOAc (100 mL) and washed with water (30 mL) and brine (30 mL). The combined aqueous layer was back-extracted with EtOAc (50 mL). The combined organic layer was dried over MgsO₄, filtered, and evaporated. The residue was purified by flash column chromatography on silica gel (80 g, gradient: EtOAc/MeOH 100/0 to 96/4) to give Intermediate 32 (1.11 g, yield: 70%) as a colorless paste.

### Intermediate 33

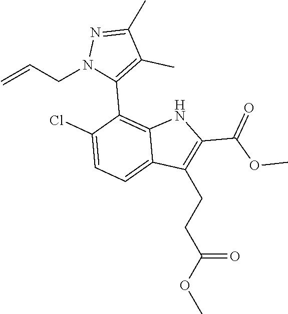 [SMILES: C=CCn1nc(C)c(C)c1-c1c(Cl)ccc2c(CCC(=O)OC)c(C(=O)OC)[nH]c12]

P(nBu)₃ (1.34 mL, 2.4 eq.) was added dropwise to a solution of Intermediate 32 (1 g, 2.23 mmol) and 2-nitrophenyl selenocyanate (1 g, 2 eq.) in anhydrous THF (20 mL) under nitrogen atmosphere. The reaction mixture was stirred at room temperature for 30 min. Hydrogen peroxide (30%, 2.23 mL, 10 eq.) was added and stirring was continued at room temperature for 16 h. The reaction mixture was diluted with EtOAc (50 mL) and water (25 mL). The organic layer was separated and the aqueous one was extracted with EtOAc (2×25 mL). The combined organic layer was dried over MgSO₄, filtered, and evaporated. The crude product was purified by flash column chromatography on silica gel (80 g, gradient: DCM/MeOH 100/0 to 98/2) followed by another purification by flash column chromatography on silica gel (40 g, gradient: heptane/EtOAc 100/0 to 40/60) to afford Intermediate 33 (355 mg, yield: 37%) as a yellow paste (approx. 80% pure).

### Intermediate 34

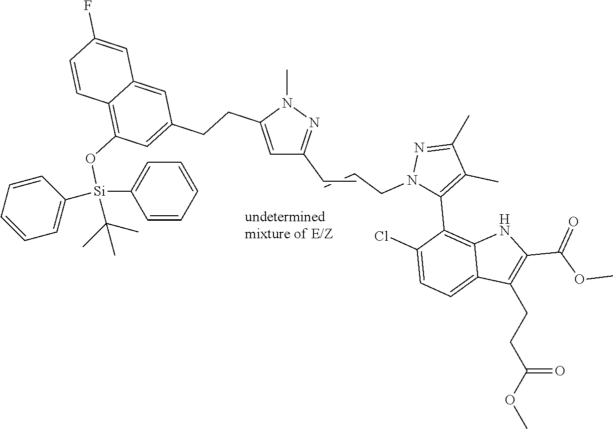

A 10 mL microwave vial was charged with Intermediate 33 (290 mg, 0.675 mmol), Intermediate 21 (595 mg, 1.5 eq.) and Pd(tBu₃P)₂ (69 mg, 0.2 eq.) under nitrogen atmosphere. The vial was sealed and DMF (8 mL) was added via syringe. The solution was degassed by bubbling nitrogen for a few min, then DIPEA (344 μL, 3 eq.) was added and the reaction mixture was heated to 120° C. in an oil bath for 3 h. The reaction mixture was diluted with EtOAc (60 mL) and water (30 mL). The organic layer was separated and washed with brine (3×25 mL). The combined aqueous layer was back-extracted with EtOAc (50 mL). The combined organic layer was dried over MgSO₄, filtered, and evaporated. The residue was purified by flash chromatography on silica gel (80 g, gradient: DCM/MeOH 100/0 to 95/5) to give Intermediate 34 (404 mg, yield: 64%) as a yellow paste (approximately 90% pure).

### Intermediate 35

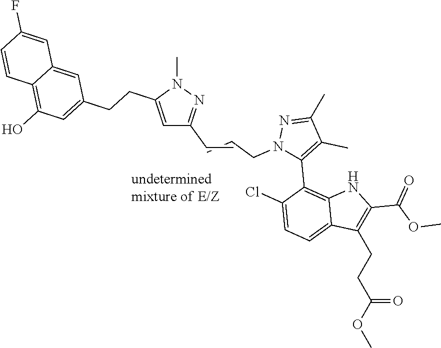

TBAF (1 M in THF, 1.15 mL, 2 eq.) was added to a solution of Intermediate 34 (540 mg, 0.577 mmol) in anhydrous THF (10 mL) stirring at room temperature under nitrogen atmosphere. The reaction mixture was stirred at room temperature for 3 h. The reaction mixture was concentrated under reduced pressure and the residue was dissolved in EtOAc (50 mL) and washed with water (25 mL) and brine (25 mL). The combined aqueous layer was back-extracted with EtOAc (25 mL). The combined organic layer was dried over MgsO₄, filtered, and evaporated. The crude product was purified by flash column chromatography on silica gel (40 g, gradient: DCM/MeOH 100/0 to 96/4) to give Intermediate 35 (220 mg, yield: 55%) as a yellowish foam.

### Intermediate 36

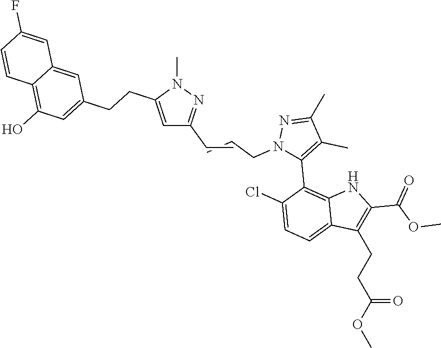

A solution of Intermediate 35 (220 mg, 0.315 mmol) in MeOH (10 mL) was hydrogenated with Pd/C (10%, 34 mg, 0.1 eq.) as the catalyst while stirring at room temperature for 2 h. The reaction mixture was filtered over a pad of Dicalite®, washing with MeOH, and the filtrate was evaporated under reduced pressure to give Intermediate 36 (190 mg, yield: 86%), used without further purification.

### Intermediate 37

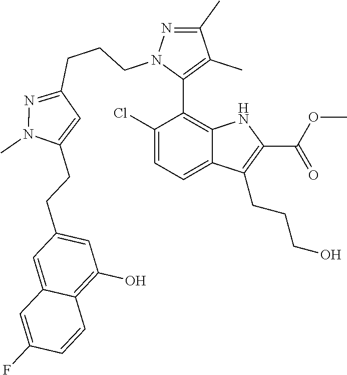

Borane dimethyl sulfide complex (2 M in TIE, 0.678 mL, 5 eq) was added to a solution of Intermediate 36 (190 mg, 0.271 mmol) in anhydrous THF (4 mL) under nitrogen atmosphere. The reaction mixture was stirred at 50° C. for 4 h. The reaction mixture was cooled to room temperature and was treated with MeOH (10 mL, added dropwise initially) and HCl (1 N, 3 mL). The mixture was stirred at room temperature for 16 h. Volatiles were removed under reduced pressure and the residue was dissolved in DCM (30 mL) and water (5 mL). The organic layer was separated and the aqueous one was extracted with DCM (2×10 mL). The combined organic layer was dried over MgSO₄, filtered, and evaporated. The residue was purified by flash column chromatography on silica gel (24 g, gradient: DCM/MeOH 100/0 to 97/3) to afford Intermediate 37 (132 mg, yield: 72%) as a foamy white solid.

### Intermediate 38

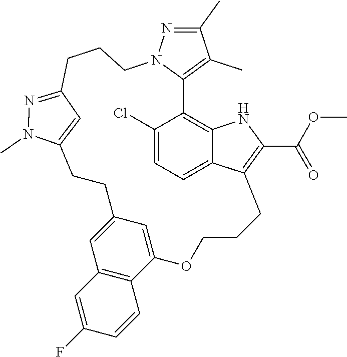

A solution of Intermediate 37 (130.0 mg, 0.194 mmol) and DTBAD (89 mg, 2 eq.) in toluene (3.9 mL) and THF (0.65 mL) was added with a syringe pump (0.1 mL/min) to a solution of PPh₃ (101 mg, 2 eq.) in toluene (3.9 mL), stirring at 70° C. Once the addition was complete, the reaction mixture was allowed to cool to room temperature. Volatiles were removed under reduced pressure and the residue was purified by flash column chromatography on silica gel (24 g, gradient: DCM/MeOH 100/0 to 96/4) to give Intermediate 38 (57 mg, yield: 45%) as a foamy white solid.

### Intermediate 39 and Intermediate 40

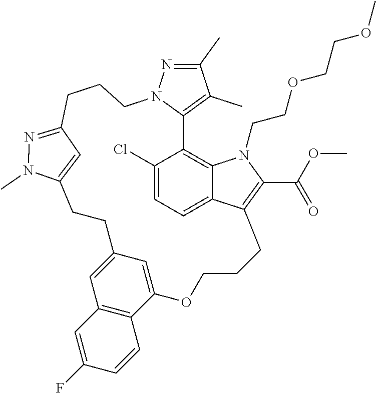

Intermediate 39: Rₐ or Sₐ; one atropisomer but absolute stereochemistry undetermined
Intermediate 40: Sₐ or Rₐ; one atropisomer but absolute stereochemistry undetermined

1-Bromo-2-(2-methoxyethoxy)ethane (15 μL, 1.3 eq.) was added to a suspension of Intermediate 38 (57 mg, 0.087 mmol) and Cs₂CO₃ (43 mg, 1.5 eq.) in anhydrous DMF (1 mL). The reaction mixture was stirred at 50° C. for 20 h. The reaction mixture was diluted with EtOAc (20 mL) and water (10 mL). The aqueous layer was separated and the organic one was washed with brine (3×5 mL). The combined aqueous layer was back-extracted with EtOAc (10 mL). The combined organic layer was dried over MgSO₄, filtered, and evaporated. The residue was purified by flash column chromatography on silica gel (12 g, gradient: DCM/MeOH 100/0 to 96/4) to afford the mixture of Intermediate 39 and Intermediate 40 as a foamy white solid. This solid was separated into its atropisomers by preparative SFC (Stationary phase: Chiralpak Diacel AD 20×250 mm, Mobile phase: CO₂, iPrOH+0.4% iPrNH₂) to afford Intermediate 39 (19 mg, yield: 29%) and Intermediate 40 (19 mg, yield: 29%).

### Intermediate 41

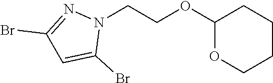 [SMILES: Brc1cc(Br)n(CCOC2CCCCO2)n1]

K₂CO₃ (22.03 g, 159 mmol, 3 eq.) was added at room temperature to a mixture of 3,5-dibromo-1H-pyrazole (CAS [67460-86-0], 12 g, 53.13 mmol) in DMF (200 mL). The reaction mixture was stirred at 60° C. and 2-(2-bromoethoxy)tetrahydro-2H-pyran (CAS #17739-45-6, 16.66 g, 1.5 eq.) was added. The reaction mixture was stirred at 100° C. for 4 h. The reaction mixture was filtered and the filtrate was washed with brine (200 mL×3). The organic layer was dried with Na₂SO₄, filtered, and concentrated. The residue was purified by flash column chromatography over silica gel (eluent: petroleum ether/EtOAc from 100/0 to 90/10) to give Intermediate 41 (16 g, yield: 85%) as a colourless oil.

### Intermediate 42

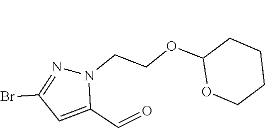

BuLi (2.5 M in THE, 25.4 mL, 63.55 mmol, 1.5 eq.) was added dropwise to a solution of Intermediate 41 (15 g, 42.37 mmol) in anhydrous THF (150 mL) at −78° C. The reaction mixture was stirred at −78° C. for 1 h before addition of DMF (9.29 g, 127.10 mmol, 3 eq.). The reaction mixture was then stirred for an additional 1 h at −78° C. The reaction was quenched by addition of aqueous NH₄Cl (100 mL) and the mixture was extracted with EtOAc (200 mL×3). The organic layer was dried over Na₂SO₄, filtered, and evaporated. The residue was purified by column chromatography on silica gel (eluent: petroleum ether/EtOAc=100/0 to 80/20) to give Intermediate 42 (6 g, yield: 47%) as a clear oil.

### Intermediate 43

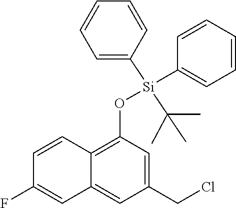 [SMILES: CC(C)(C)[Si](Oc1cc(CCl)cc2cc(F)ccc12)(c1ccccc1)c1ccccc1]

DIPEA (1.12 mL, 6.84 mmol, 3 eq.) was added to a solution of Intermediate 17 (1 g, 2.278 mmol) and methanesulfonic anhydride (0.794 g, 4.56 mmol, 2 eq.) in THE (200 mL) at 0° C. The reaction mixture was stirred at room temperature overnight. The reaction mixture was cooled to 0° C. and LiCl (0.386 g, 9.113 mmol, 4 eq.) was added. Stirring was continued at room temperature for 2 h. The reaction was quenched by addition of water (20 mL), and the mixture was extracted with EtOAc (20 mL×3). The combined organic layer was dried with Na₂SO₄, filtered, and evaporated. The residue was purified by column chromatography on silica gel (eluent: petroleum ether/EtOAc=100/0 to 5/1) to give Intermediate 43 (450 mg, yield: 44%) as a white solid and its corresponding mesylate (450 mg, yield: 39%) as a white solid.

### Intermediate 44

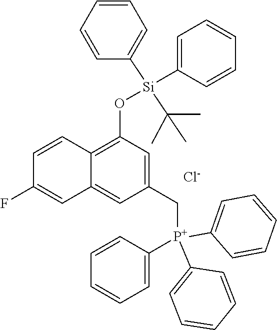 [SMILES: CC(C)(C)[Si](Oc1cc(C[P+](c2ccccc2)(c2ccccc2)c2ccccc2)cc2cc(F)ccc12)(c1ccccc1)c1ccccc1 ; [Cl-]]

PPh₃ (680 mg, 2.592 mmol, 1.2 eq.) was added to a solution of Intermediate 43 (970 mg, 2.16 mmol) in ACN (10 mL). The reaction mixture was stirred at 85° C. for 16 h. The solvent was evaporated and the residue was triturated in petroleum ether (20 mL) at room temperature. The solid was filtered and dried under vacuum to give Intermediate 44 (1.1 g, yield: 70%) as a white solid.

### Intermediate 45

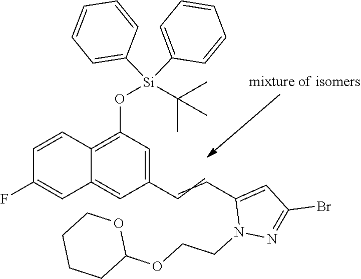 [SMILES: CC(C)(C)[Si](Oc1cc(C=Cc2cc(Br)nn2CCOC2CCCCO2)cc2cc(F)ccc12)(c1ccccc1)c1ccccc1]

NaH (60% in mineral oil, 887 mg, 22.17 mmol, 1.2 eq.) was added to a solution of Intermediate 44 (15.04 g, 20.32 mmol, 1.1 eq.) in THF (150 mL) at 0° C. The reaction mixture was stirred at 0° C. for 1 h. Intermediate 42 (5.6 g, 18.47 mmol) was then added at −30° C. and the reaction mixture was stirred at −30° C. for 2 h. The reaction was quenched by addition of aqueous NH₄Cl (50 mL) and the mixture was extracted with EtOAc (100 mL×3). The combined organic layer was separated, dried with Na₂SO₄, filtered, and evaporated. The residue was purified by column chromatography on silica gel (eluent: petroleum ether/EtOAc=100/0 to 80/20) to give Intermediate 45 (12 g, yield: 93%) as a white solid.

### Intermediate 46

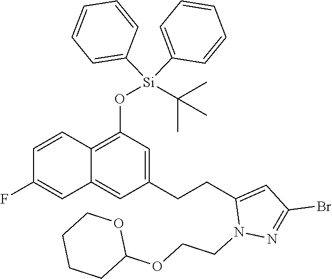 [SMILES: CC(C)(C)[Si](Oc1cc(CCc2cc(Br)nn2CCOC2CCCCO2)cc2cc(F)ccc12)(c1ccccc1)c1ccccc1]

NaHCO₃ (2.345 g, 28.58 mmol, 2 eq.) was added to a solution of Intermediate 45 (10 g, 14.29 mmol) and p-toluenesulfonyl hydrazide (9.315 g, 50.01 mmol, 3.5 eq.) in a mixture of THF and water (4/1, 70 mL). The reaction mixture was stirred at 80° C. for 7 h. The solvent was evaporated. The residue was purified by column chromatography on silica gel (eluent: petroleum ether/EtOAc=100/0 to 80/20) to give Intermediate 46 (6.905 g, yield: 69%) as a white solid.

### Intermediate 47

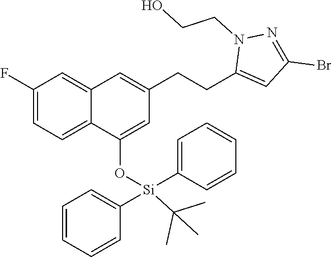 [SMILES: CC(C)(C)[Si](Oc1cc(CCc2cc(Br)nn2CCO)cc2cc(F)ccc12)(c1ccccc1)c1ccccc1]

PTSA (68 mg, 0.356 mmol, 0.25 eq.) was added to a suspension of Intermediate 46 (1 g, 1.425 mmol) in MeOH (20 mL). The reaction mixture was stirred at room temperature for 3 h. The solvent was evaporated and the residue was dissolved in DCM (50 mL), and this solution was washed with saturated aqueous NaHCO₃ (2×10 mL). The organic layer was dried over MgSO₄, filtered, and evaporated to give Intermediate 47 (assumed quantitative), used directly in the next step.

### Intermediate 48

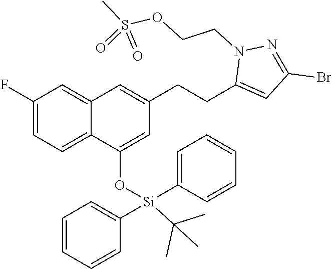 [SMILES: CC(C)(C)[Si](Oc1cc(CCc2cc(Br)nn2CCOS(C)(=O)=O)cc2cc(F)ccc12)(c1ccccc1)c1ccccc1]

A solution of Intermediate 47 (crude, 780 mg, 1.623 mmol) and Et₃N (263 μL, 1.894 mmol, 1.5 eq.) in DCM (15 mL) was cooled down to 0° C. MsCl (123 μL, 1.579 mmol, 1.25 eq.) was added and the reaction mixture was then stirred at room temperature for 30 min. The reaction mixture was diluted with DCM (30 mL) and saturated aqueous NaHCO₃ (10 mL). The organic layer was separated, dried over MgSO₄, filtered, and evaporated to give Intermediate 48 (878 mg, yield assumed quantitative), used without further purification.

### Intermediate 49

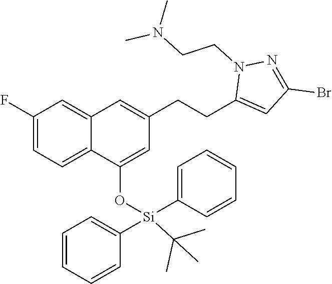 [SMILES: CN(C)CCn1nc(Br)cc1CCc1cc(O[Si](c2ccccc2)(c2ccccc2)C(C)(C)C)c2ccc(F)cc2c1]

A solution of Intermediate 48 (crude, 878 mg, 1.262 mmol) and dimethylamine (2 M in TI-F, 15.8 mL, 31.55 mmol, 25 eq.) in a closed vessel was stirred at 50° C. for 16 h. The reaction mixture was diluted with EtOAc (50 mL) and water (25 mL). The organic layer was separated and the aqueous one was back-extracted with EtOAc (25 mL). The combined organic layer was dried over MgSO₄, filtered and evaporated. The residue was purified by flash column chromatography on silica gel (40 g, gradient: from heptane/EtOAc 100/0 to 0/100) to obtain Intermediate 49 (725 mg, yield: 89%) as a colorless paste.

### Intermediate 50

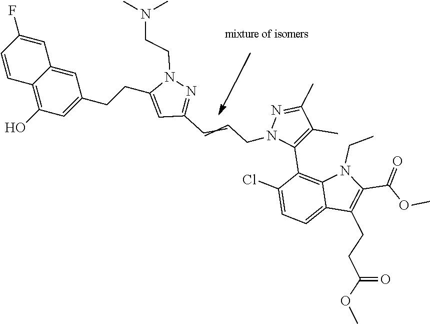 [SMILES: CCn1c(C(=O)OC)c(CCC(=O)OC)c2ccc(Cl)c(-c3c(C)c(C)nn3CC=Cc3cc(CCc4cc(O)c5ccc(F)cc5c4)n(CCN(C)C)n3)c21]

A vial was charged with Intermediate 25 (250 mg, 0.546 mmol), Intermediate 49 (493 mg, 0.764 mmol, 1.4 eq.) and bis(tri-tert-butylphosphine)palladium(0) (CAS [53199-31-8], 56 mg, 0.109 mmol, 0.2 eq.) under nitrogen atmosphere. The vial was sealed and DMF (6 mL) was added via syringe. The solution was degassed by bubbling nitrogen through for a few minutes, then DIPEA (280 μL, 1.64 mmol, 3 eq.) was added and the reaction mixture was stirred at 120° C. for 4 h. The reaction mixture was cooled to room temperature and was diluted with EtOAc (50 mL) and water (30 mL). The organic layer was separated and washed with brine (3×15 mL). The combined aqueous layer was back-extracted with EtOAc (30 mL). The combined organic layer was dried over MgSO₄, filtered, and evaporated. The residue was dissolved in anhydrous THE (10 mL) and TBAF (1 M in THF, 1.53 mL, 1.53 mmol, 2 eq.) was added while stirring at room temperature under nitrogen atmosphere. The reaction mixture was stirred at room temperature for 1 h. The reaction mixture was concentrated under reduced pressure. The residue was dissolved in EtOAc (50 mL) and washed with water (25 mL) and brine (25 mL). The combined aqueous layer was back-extracted with EtOAc (25 mL). The combined organic layer was dried over MgSO₄, filtered, and evaporated. The residue was purified by flash column chromatography on silica gel (40 g, gradient: DCM/MeOH (NH₃) 100/0 to 96/4) to afford Intermediate 50 (325 mg, 54%) as a white foam.

### Intermediate 51

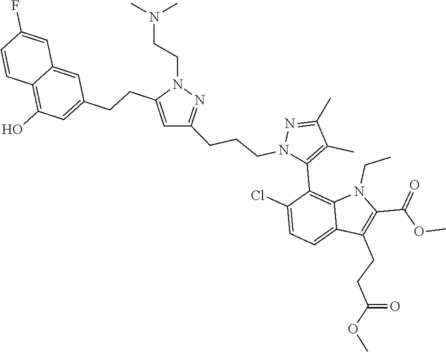 [SMILES: CCn1c(C(=O)OC)c(CCC(=O)OC)c2ccc(Cl)c(-c3c(C)c(C)nn3CCCc3cc(CCc4cc(O)c5ccc(F)cc5c4)n(CCN(C)C)n3)c21]

A solution of Intermediate 50 (325 mg, 0.415 mmol) in MeOH (40 mL) was hydrogenated at atmospheric pressure in the presence of Pd/C (10%, 44 mg, 0.1 eq.) as catalyst while stirring for 3 h at room temperature. The reaction mixture was filtered over dicalite, washing with MeOH, and the filtrate was evaporated under reduced pressure to give Intermediate 51 (275 mg, yield: 84%) as a yellow foam, used without further purification.

### Intermediate 52

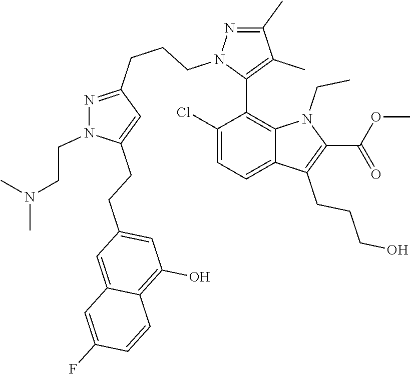

Borane dimethyl sulfide complex (2 M in THF, 0.828 mL, 1.655 mmol, 4 eq.) was added to a solution of Intermediate 51 (325 mg, 0.415 mmol) in anhydrous THE (5 mL) under nitrogen atmosphere. The reaction mixture was stirred at 50° C. for 4 h. Additional borane dimethyl sulphide complex (0.415 mL, 0.83 mmol, 2 eq.) was added and the reaction mixture was stirred for a further 2 h. Another batch of borane dimethyl sulphide complex (0.415 mL, 0.83 mmol, 2 eq.) was added and the reaction was stirred for 1 h. The reaction mixture was cooled to room temperature and treated with MeOH (10 mL, added dropwise initially) and aqueous HCl (1 N, 3 mL). The mixture was stirred at room temperature for 16 h. Volatiles were removed under reduced pressure. The residue was dissolved in DCM (40 mL) and water (20 mL). The organic layer was separated and the aqueous one was extracted with DCM (2×20 mL). The combined organic layer was dried over MgSO₄, filtered, and evaporated. The residue was purified by flash column chromatography on silica gel (40 g, gradient: DCM/MeOH (NH₃) 100/0 to 96/4) to give a white solid (borane complex). This solid was dissolved in HCl (1.25 M in MeOH) in a closed vessel and stirred at 50° C. for 16 h. Volatiles were removed under reduced pressure and the residue was partitioned between saturated aqueous NaHCO₃ (5 mL) and DCM (20 mL). The organic layer was separated and the aqueous one was extracted with DCM (2×10 mL). The combined organic layer was dried over MgSO₄, filtered, and evaporated to obtain Intermediate 52 (90 mg, yield: 29%) as a white solid.

### Intermediate 53 and Intermediate 54

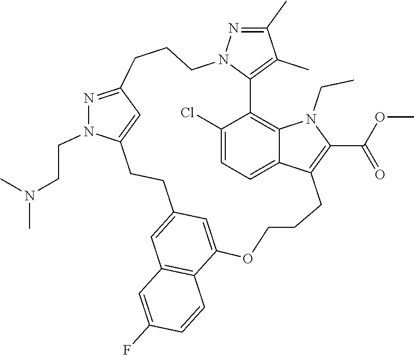

Intermediate 53: Rₐ or Sₐ; one atropisomer but absolute stereochemistry undetermined
Intermediate 54: Sₐ or Rₐ; one atropisomer but absolute stereochemistry undetermined

A solution of Intermediate 52 (90 mg, 0.119 mmol) and di-tert-butyl azodicarboxylate (82.1 mg, 0.357 mmol, 3 eq.) in toluene (2.5 mL) and THF (0.6 mL) was added with a syringe pump (0.1 mL/min) to a solution of triphenylphosphine (93 mg, 0.357 mmol, 3 eq.) in toluene (2.5 mL) stirring at 70° C. Once the addition was complete, the reaction mixture was allowed to cool down to room temperature and volatiles were removed under reduced pressure. The residue was purified by flash column chromatography on silica gel (24 g, gradient: DCM/MeOH (NH₃) 100/0 to 95/5) followed by preparative SFC (Stationary phase: Chiralpak Diacel AD 20×250 mm, Mobile phase: CO₂, EtOH+0.4% iPrNH₂) to give Intermediate 53 (20 mg, yield: 23%) and Intermediate 54 (20 mg, yield: 23%).

### Intermediate 55

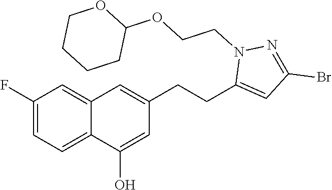 [SMILES: Oc1cc(CCc2cc(Br)nn2CCOC2CCCCO2)cc2cc(F)ccc12]

TBAF (1 M in THF, 2.99 mL, 2.993 mmol, 1.4 eq.) was added to a solution of Intermediate 46 (1.50 g, 2.138 mmol) in anhydrous THE (40 mL) stirring under nitrogen atmosphere at room temperature. The reaction mixture was stirred at room temperature for 16 h. Volatiles were removed under reduced pressure and the residue was dissolved in DCM (25 mL) and washed with water (10 mL). The organic layer was dried over MgSO₄, filtered, and evaporated to afford Intermediate 55 (990 mg, yield assumed quantitative), used without further purification in the following step.

### Intermediate 56

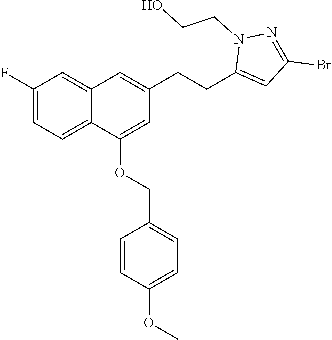 [SMILES: COc1ccc(COc2cc(CCc3cc(Br)nn3CCO)cc3cc(F)ccc23)cc1]

NaH (60% dispersion in mineral oil, 119.6 mg, 2.991 mmol, 1.4 eq.) was added to a solution of Intermediate 55 (crude for previous step, 990 mg, 2.137 mmol) in dry DMF (25 mL) stirring at 0° C. under nitrogen atmosphere. After 5 minutes, 4-methoxybenzyl chloride (0.435 mL, 3.205 mmol, 1.5 eq.) was added dropwise. The reaction mixture was stirred at room temperature for 2 h. The reaction was quenched by addition of water (50 mL) and diluted with EtOAc (100 mL). The organic layer was separated and washed with brine (2×50 mL). The combined aqueous layer was back-extracted with EtOAc (50 mL). The combined organic layer was dried over MgSO₄, filtered and evaporated. The residue was purified by flash chromatography on silica gel (40 g, gradient: heptane/EtOAc 100/0 to 0/100). The obtained product (mixture of Intermediate 56 and its THP-protected precursor) was dissolved in MeOH (10 mL) and the solution was cooled down to 0° C. HCl (1.25 M in MeOH, 8.6 mL, 10.7 mmol, 5 eq.) was added and the reaction mixture was stirred at room temperature for 1 h. A precipitate formed and was filtered, washed with ice-cold MeOH (10 mL), and dried in vacuo to afford Intermediate 56 (755 mg, yield: 70%) as a off-white solid.

### Intermediate 57

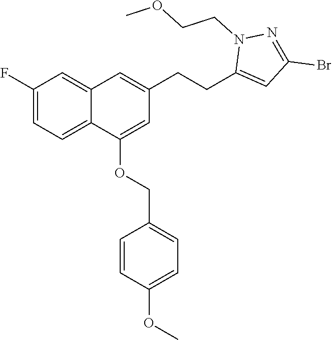 [SMILES: COCCn1nc(Br)cc1CCc1cc(OCc2ccc(OC)cc2)c2ccc(F)cc2c1]

A solution of Intermediate 56 (755 mg, 1.512 mmol) in dry DMF (6 mL) was cooled down to 0° C. under nitrogen atmosphere. NaH (60% dispersion in mineral oil, 91 mg, 2.267 mmol, 1.5 eq.) was added and, after 10 minutes, Mel (0.188 mL, 3.023 mmol, 2 eq.) was added. The reaction mixture was stirred at room temperature for 16 h. The reaction mixture was diluted with EtOAc (50 mL) and water (30 mL). The aqueous layer was separated and the organic one was washed with brine (2×30 mL). The combined aqueous layer was back-extracted with EtOAc (30 mL). The combined organic layer was dried over MgSO₄, filtered, and evaporated. The residue was purified by flash column chromatography on silica gel (40 g, heptane/EtOAc 100/0 to 70/30) to afford Intermediate 57 (735 mg, yield: 94%) as a white solid.

### Intermediate 58

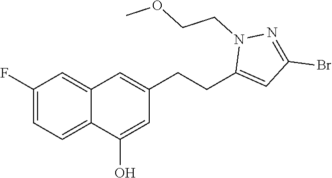 [SMILES: COCCn1nc(Br)cc1CCc1cc(O)c2ccc(F)cc2c1]

Triflic acid (547 μL, 6.184 mmol) was added dropwise to a solution of Intermediate 57 (635 mg, 1.236 mmol) in 1,3-dimethoxybenzene (10 mL) and DCM (10 mL) stirring at 0° C. The reaction mixture was stirred for 15 min at 0° C. The reaction mixture was diluted with DCM (30 mL) and treated with saturated aqueous NaHCO₃ (20 mL). The organic layer was separated and the aqueous one was extracted with DCM (10 mL). The combined organic layer was dried over MgSO₄, filtered, and evaporated. The residue was purified by flash column chromatography on silica gel (40 g, gradient: heptane/EtOAc 100/0 to 50/50) to afford Intermediate 58 (462 mg, yield: 77%) as a yellow paste.

### Intermediate 59

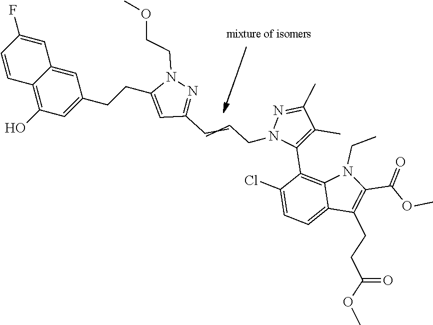 [SMILES: CCn1c(C(=O)OC)c(CCC(=O)OC)c2ccc(Cl)c(-c3c(C)c(C)nn3CC=Cc3cc(CCc4cc(O)c5ccc(F)cc5c4)n(CCOC)n3)c21]

A vial was charged with Intermediate 25 (300 mg, 0.655 mmol), Intermediate 58 (354 mg, 80% pure, 0.721 mmol, 1.1 eq.) and bis(tri-tert-butylphosphine)palladium(0) (67 mg, 0.131 mmol, 0.2 eq.) under nitrogen atmosphere. The vial was sealed and DMF (7.5 mL) was added via syringe. The solution was degassed by bubbling nitrogen through for a few min, then DIPEA (334 μL, 1.965 mmol, 3 eq.) was added and the reaction mixture was stirred at 120° C. for 2 h. The reaction mixture was cooled down to room temperature and diluted with EtOAc (50 mL) and water (30 mL). The layers were separated and the organic one was washed with brine (2×25 mL). The combined aqueous layer was back-extracted with EtOAc (30 mL). The combined organic layer was dried over MgSO₄, filtered, and evaporated. The residue was purified by flash chromatography on silica gel (80 g, gradient: DCM/MeOH 100/0 to 96/4) to afford Intermediate 59 (300 mg, yield: 59%).

### Intermediate 60

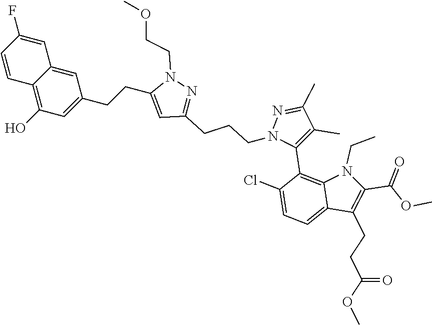 [SMILES: CCn1c(C(=O)OC)c(CCC(=O)OC)c2ccc(Cl)c(-c3c(C)c(C)nn3CCCc3cc(CCc4cc(O)c5ccc(F)cc5c4)n(CCOC)n3)c21]

A solution of Intermediate 59 (400 mg, 0.633 mmol) in MeOH (20 mL) was hydrogenated under atmospheric pressure with Pd/C (10%, 22 mg, 0.1 eq.) as catalyst while stirring at room temperature for 5 h. The reaction mixture was filtered over dicalite, washing with MeOH and the filtrate was evaporated under reduced pressure. The residue was purified by flash column chromatography on silica gel (40 g, gradient: DCM/MeOH 100/0 to 96/4) to give Intermediate 60 (315 mg, 78%) as a foamy white solid.

### Intermediate 61

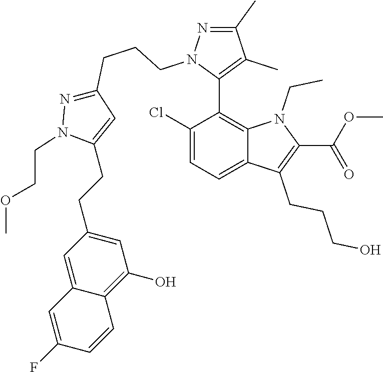 [SMILES: CCn1c(C(=O)OC)c(CCCO)c2ccc(Cl)c(-c3c(C)c(C)nn3CCCc3cc(CCc4cc(O)c5ccc(F)cc5c4)n(CCOC)n3)c21]

Borane dimethyl sulfide complex (2 M in THF, 1.02 mL, 2.04 mmol, 5 eq.) was added to a solution of Intermediate 60 (315 mg, 0.408 mmol) in anhydrous THF (5 mL) under nitrogen atmosphere. The reaction mixture was stirred at 50° C. for 5 h. After cooling to room temperature, the reaction mixture was treated with MeOH (10 mL, added dropwise initially) and aqueous HCl (1 N, 3 mL) to break the BH₃ complex. The mixture was stirred at room temperature for 16 h. Volatiles were removed under reduced pressure and the residue was dissolved in DCM (30 mL) and water (20 mL). The layers were separated and the aqueous one was extracted with DCM (20 mL). The combined organic layer was dried over MgSO₄, filtered, and evaporated. The residue was purified by flash column chromatography on silica gel (40 g, gradient: DCM/MeOH 100/0 to 96/4) to afford Intermediate 61 (210 mg, yield: 69%) as a foamy white solid.

### Intermediate 62 and Intermediate 63

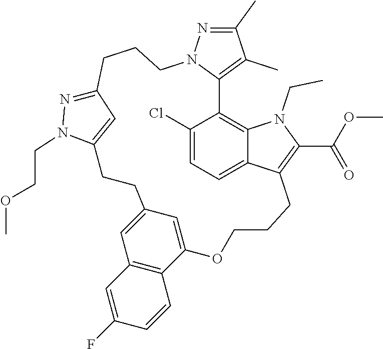 [SMILES: CCn1c(C(=O)OC)c2c3ccc(Cl)c(c31)-c1c(C)c(C)nn1CCCc1cc(n(CCOC)n1)CCc1cc(c3ccc(F)cc3c1)OCCC2]

Intermediate 62: Rₐ or Sₐ; one atropisomer but absolute stereochemistry undetermined
Intermediate 63: Sₐ or Rₐ; one atropisomer but absolute stereochemistry undetermined A solution of Intermediate 61 (210 mg, 0.282 mmol) and di-tert-butyl azodicarboxylate (130 mg, 0.564 mmol, 2 eq.) in toluene (5.5 mL) and THF (2.0 mL) was added with a syringe pump (0.1 mL/min) to a solution of triphenylphosphine (148 mg, 0.564 mmol, 2 eq.) in toluene (5.5 mL) stirring at 70° C. Once the addition was complete, the reaction mixture was cooled to room temperature and volatiles were removed under reduced pressure. The residue was purified by flash column chromatography on silica gel (40 g, gradient: DCM/MeOH(NH₃) 100/0 to 97/3) followed by preparative SFC (Stationary phase: Chiralpak Diacel AD 20×250 mm, Mobile phase: CO₂, EtOH+0.4% iPrNH₂) to give Intermediate 62 (48 mg, yield: 23%) and Intermediate 63 (48 mg, yield: 23%).

### Preparation of Compounds

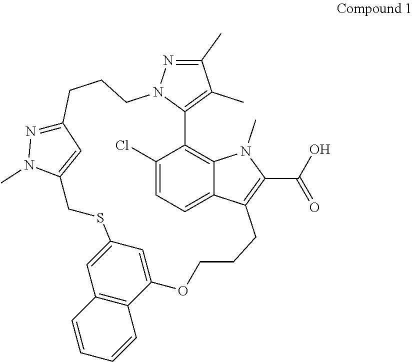 [SMILES: Cc1nn2c(c1C)-c1c(Cl)ccc3c(c(C(=O)O)n(C)c13)CCCOc1cc(cc3ccccc13)SCc1cc(nn1C)CCC2]

Rₐ or Sₐ; one atropisomer but absolute stereochemistry undetermined

A solution of LiOH (32 mg, 15 eq.) in water (1 mL) was added to a solution of Intermediate 14 (60 mg, 0.09 mmol) in a mixture of THF (2 mL) and MeOH (2 mL). The reaction mixture was heated at 60° C. for 3 h. The mixture was cooled to room temperature, diluted with MeOH and directly injected into preparative HPLC (Stationary phase: RP XBridge Prep C18 OBD-10 μm, 30×150 mm, Mobile phase: 0.25% NH₄HCO₃ solution in water, CH₃CN) to afford Compound 1 (41 mg, yield: 70%) as an off-white solid.

¹H NMR (400 MHz, DMSO-d₆) δ ppm 1.27-1.38 (m, 1H), 1.56-1.67 (m, 1H), 1.70 (s, 3H), 2.07-2.13 (m, 2H), 2.14 (s, 3H), 2.28-2.37 (m, 2H), 3.06-3.15 (m, 1H), 3.31 (s, 3H), 3.38-3.57 (m, 3H), 3.69 (s, 3H), 3.92-4.00 (m, 1H), 4.08-4.17 (m, 1H), 4.19-4.25 (m, 1H), 4.25-4.31 (m, 1H), 4.72 (s, 1H), 6.79 (d, J=1.1 Hz, 1H), 7.20 (d, J=8.6 Hz, 1H), 7.25 (s, 1H), 7.38-7.48 (m, 2H), 7.64-7.68 (m, 1H), 7.98-8.04 (m, 2H).

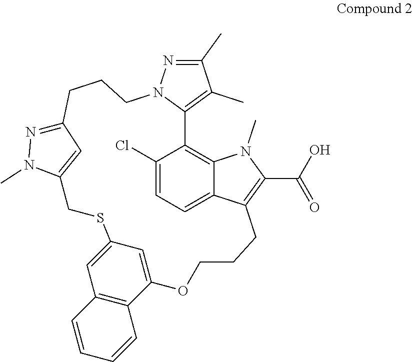 [SMILES: Cc1nn2c(c1C)-c1c(Cl)ccc3c(c(C(=O)O)n(C)c13)CCCOc1cc(cc3ccccc13)SCc1cc(nn1C)CCC2]

Sₐ or Rₐ; one atropisomer but absolute stereochemistry undetermined

Compound 2 was prepared according to an analogous procedure as for Compound 1, starting from Intermediate 15 instead of Intermediate 14.

¹H NMR (400 MHz, DMSO-d₆) δ ppm 1.26-1.39 (m, 1H), 1.55-1.67 (m, 1H), 1.70 (s, 3H), 2.07-2.12 (m, 2H), 2.14 (s, 3H), 2.27-2.37 (m, 2H), 3.06-3.15 (m, 1H), 3.31 (s, 3H), 3.37-3.56 (m, 3H), 3.70 (s, 3H), 3.92-4.00 (m, 1H), 4.09-4.17 (m, 1H), 4.19-4.25 (m, 1H), 4.25-4.31 (m, 1H), 4.72 (s, 1H), 6.79 (d, J=1.1 Hz, 1H), 7.20 (d, J=8.6 Hz, 1H), 7.26 (s, 1H), 7.38-7.48 (m, 2H), 7.64-7.69 (m, 1H), 7.98-8.04 (m, 2H).

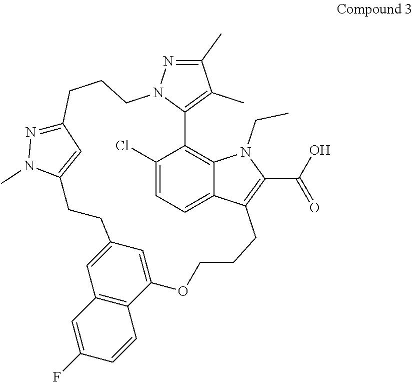 [SMILES: CCn1c(C(=O)O)c2c3ccc(Cl)c(c31)-c1c(C)c(C)nn1CCCc1cc(n(C)n1)CCc1cc(c3ccc(F)cc3c1)OCCC2]

Rₐ or Sₐ; one atropisomer but absolute stereochemistry undetermined

Compound 3 was prepared according to an analogous procedure as for Compound 1, starting from Intermediate 30 instead of Intermediate 14.

¹H NMR (400 MHz, DMSO-d₆) δ ppm 0.84 (t, J=6.9 Hz, 3H), 1.38-1.51 (m, 1H), 1.55-1.67 (m, 1H), 1.78 (s, 3H), 2.08 (br t, J=7.3 Hz, 2H), 2.16 (s, 3H), 2.18-2.30 (m, 2H), 2.82-2.93 (m, 2H), 2.94-3.07 (m, 2H), 3.11-3.27 (m, 2H), 3.39-3.55 (m, 6H), 3.79-3.96 (m, 2H), 4.35-4.47 (m, 1H), 4.98 (s, 1H), 6.40 (s, 1H), 7.11-7.16 (m, 2H), 7.27 (td, J=8.9, 2.6 Hz, 1H), 7.46 (dd, J=10.5, 2.6 Hz, 1H), 7.87 (d, J=8.6 Hz, 1H), 8.11 (dd, J=9.2, 5.9 Hz, 1H).

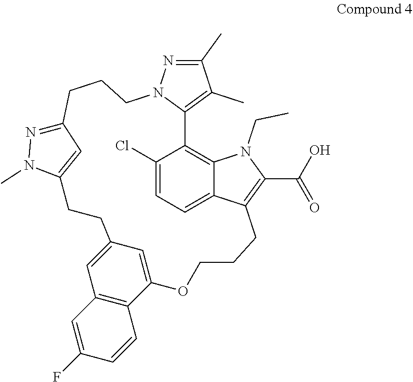 [SMILES: CCn1c(C(=O)O)c2c3ccc(Cl)c(c31)-c1c(C)c(C)nn1CCCc1cc(n(C)n1)CCc1cc(c3ccc(F)cc3c1)OCCC2]

Sₐ or Rₐ; one atropisomer but absolute stereochemistry undetermined

Compound 4 was prepared according to an analogous procedure as for Compound 1, starting from Intermediate 31 instead of Intermediate 14.

¹H NMR (400 MHz, DMSO-d₆) δ ppm 0.84 (t, J=6.9 Hz, 3H), 1.38-1.51 (m, 1H), 1.55-1.68 (m, 1H), 1.78 (s, 3H), 2.08 (br t, J=7.3 Hz, 2H), 2.16 (s, 3H), 2.19-2.29 (m, 2H), 2.82-2.94 (m, 2H), 2.94-3.06 (m, 2H), 3.11-3.27 (m, 2H), 3.40-3.56 (m, 6H), 3.79-3.96 (m, 2H), 4.35-4.47 (m, 1H), 4.97 (s, 1H), 6.40 (s, 1H), 7.11-7.17 (m, 2H), 7.27 (td, J=8.9, 2.7 Hz, 1H), 7.46 (dd, J=10.5, 2.6 Hz, 1H), 7.88 (d, J=8.6 Hz, 1H), 8.11 (dd, J=9.2, 5.9 Hz, 1H).

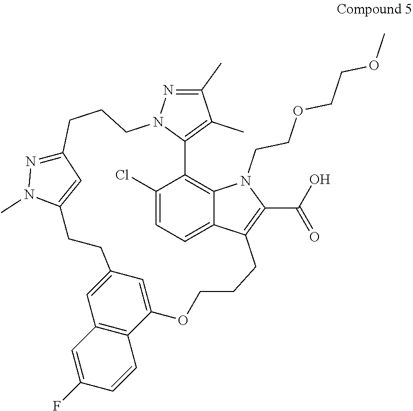

Rₐ or Sₐ; one atropisomer but absolute stereochemistry undetermined

Compound 5 was prepared according to an analogous procedure as for Compound 1, starting from Intermediate 39 instead of Intermediate 14.

¹H NMR (400 MHz, CHLOROFORM-d) δ ppm 1.68-1.80 (m, 2H), 1.90 (s, 3H), 2.04-2.31 (m, 7H), 2.83-2.96 (m, 4H), 3.15-3.29 (m, 10H), 3.30-3.37 (m, 3H), 3.48 (dt, J=15.0, 4.5 Hz, 1H), 3.52-3.69 (m, 4H), 3.82 (dt, J=14.1, 7.1 Hz, 1H), 4.59 (ddd, J=14.9, 7.7, 5.3 Hz, 1H), 5.42 (s, 1H), 5.84 (s, 1H), 7.10 (s, 1H), 7.13-7.22 (m, 2H), 7.28 (dd, J=10.2, 2.7 Hz, 1H), 7.65 (d, J=8.6 Hz, 1H), 8.25 (dd, J=9.2, 5.8 Hz, 1H).

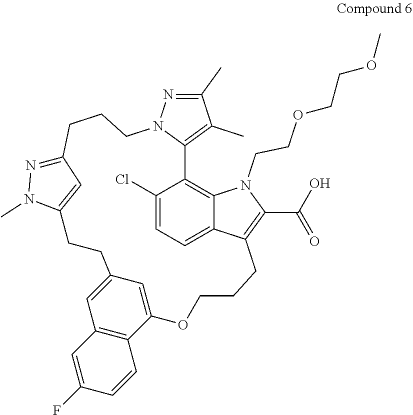

Sₐ or Rₐ; one atropisomer but absolute stereochemistry undetermined

Compound 6 was prepared according to an analogous procedure as for Compound 1, starting from Intermediate 40 instead of Intermediate 14.

¹H NMR (400 MHz, CHLOROFORM-d) δ ppm 1.69-1.80 (m, 2H), 1.89 (s, 3H), 2.04-2.32 (m, 7H), 2.83-2.96 (m, 4H), 3.16-3.30 (m, 10H), 3.30-3.36 (m, 3H), 3.47 (dt, J=15.0, 4.6 Hz, 1H), 3.52-3.68 (m, 4H), 3.81 (dt, J=14.1, 7.1 Hz, 1H), 4.59 (ddd, J=14.8, 7.8, 5.5 Hz, 1H), 5.42 (s, 1H), 5.85 (s, 1H), 7.09 (s, 1H), 7.13-7.22 (m, 2H), 7.28 (dd, J=10.1, 2.6 Hz, 1H), 7.65 (d, J=8.6 Hz, 1H), 8.25 (dd, J=9.2, 5.8 Hz, 1H).

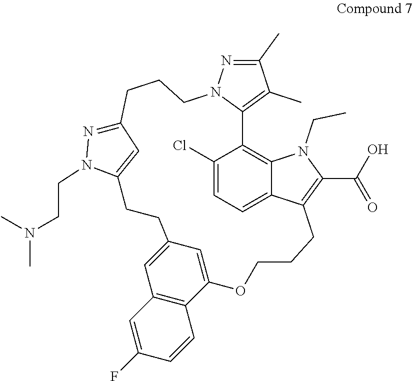 [SMILES: CCn1c(C(=O)O)c2c3ccc(Cl)c(c31)-c1c(C)c(C)nn1CCCc1cc(n(CCN(C)C)n1)CCc1cc(c3ccc(F)cc3c1)OCCC2]

Rₐ or Sₐ; one atropisomer but absolute stereochemistry undetermined

Compound 7 was prepared according to an analogous procedure as for Compound 1, starting from Intermediate 53 instead of Intermediate 14.

¹H NMR (400 MHz, DMSO-d₆) δ ppm 0.84 (t, J=7.0 Hz, 3H), 1.41-1.55 (m, 2H), 1.80 (s, 3H), 2.09-2.22 (m, 13H), 2.42-2.49 (m, 1H), 2.56-2.64 (m, 1H), 2.88-3.08 (m, 5H), 3.09-3.18 (m, 1H), 3.41-3.52 (m, 2H), 3.52-3.61 (m, 1H), 3.69-3.78 (m, 1H), 3.81-3.97 (m, 3H), 4.37-4.48 (m, 1H), 5.26 (s, 1H), 6.41 (s, 1H), 7.11 (d, J=8.5 Hz, 1H), 7.20 (s, 1H), 7.25 (td, J=8.9, 2.7 Hz, 1H), 7.47 (dd, J=10.5, 2.6 Hz, 1H), 7.75 (d, J=8.5 Hz, 1H), 8.10 (dd, J=9.2, 5.9 Hz, 1H).

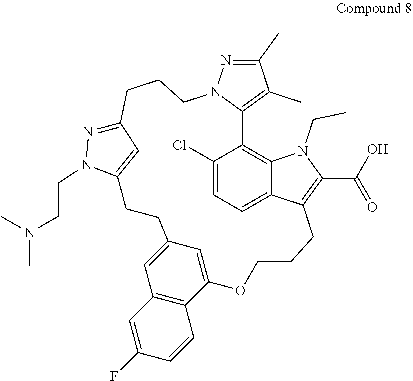 [SMILES: CCn1c(C(=O)O)c2c3ccc(Cl)c(c31)-c1c(C)c(C)nn1CCCc1cc(n(CCN(C)C)n1)CCc1cc(c3ccc(F)cc3c1)OCCC2]

Sₐ or Rₐ; one atropisomer but absolute stereochemistry undetermined

Compound 8 was prepared according to an analogous procedure as for Compound 1, starting from Intermediate 54 instead of Intermediate 14.

¹H NMR (400 MHz, DMSO-d₆) δ ppm 0.84 (t, J=7.0 Hz, 3H), 1.42-1.53 (m, 2H), 1.80 (s, 3H), 2.09-2.22 (m, 13H), 2.43-2.48 (m, 1H), 2.55-2.63 (m, 1H), 2.88-3.08 (m, 5H), 3.09-3.18 (m, 1H), 3.41-3.51 (m, 2H), 3.53-3.61 (m, 1H), 3.70-3.78 (m, 1H), 3.80-3.97 (m, 3H), 4.37-4.47 (m, 1H), 5.27 (s, 1H), 6.41 (s, 1H), 7.11 (d, J=8.5 Hz, 1H), 7.20 (s, 1H), 7.26 (td, J=8.9, 2.6 Hz, 1H), 7.47 (dd, J=10.5, 2.6 Hz, 1H), 7.74 (d, J=8.5 Hz, 1H), 8.11 (dd, J=9.2, 5.9 Hz, 1H).

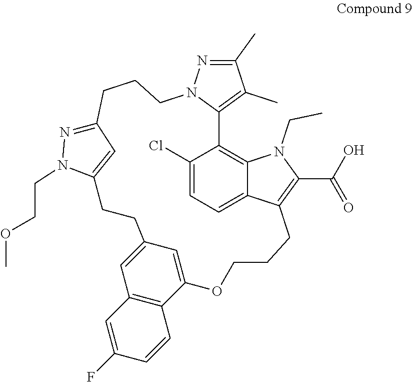 [SMILES: CCn1c(C(=O)O)c2c3ccc(Cl)c(c31)-c1c(C)c(C)nn1CCCc1cc(n(CCOC)n1)CCc1cc(c3ccc(F)cc3c1)OCCC2]

Rₐ or Sₐ; one atropisomer but absolute stereochemistry undetermined

A solution of LiOH (23.7 mg, 0.991 mmol, 15 eq.) in water (0.5 mL) was added to a solution of Intermediate 62 (48 mg, 0.0661 mmol) in THE (1 mL) and MeOH (1 mL). The reaction mixture was stirred at 60° C. for 3 h. After cooling, the reaction mixture was diluted with MeOH and this solution was purified by preparative HPLC (Stationary phase: RP XBridge Prep C18 OBD-10 μm, 30×150 mm, Mobile phase: 0.25% NH₄HCO₃ solution in water, CH₃CN) followed by flash column chromatography on silica gel (12 g, gradient: DCM/MeOH 100/0 to 92/8). The obtained solid was suspended in DIPE and evaporated to give Compound 9 (38 mg, yield: 81%) as a yellow powder.

¹H NMR (400 MHz, DMSO-d₆) δ ppm 0.84 (t, J=6.9 Hz, 3H), 1.38-1.50 (m, 1H), 1.57-1.69 (m, 1H), 1.77 (s, 3H), 2.11 (br t, J=7.3 Hz, 2H), 2.16 (s, 3H), 2.19-2.29 (m, 2H), 2.80-2.93 (m, 2H), 2.95-3.07 (m, 2H), 3.12 (s, 3H), 3.14-3.27 (m, 2H), 3.40-3.55 (m, 5H), 3.77-3.96 (m, 4H), 4.34-4.45 (m, 1H), 4.98 (s, 1H), 6.38 (s, 1H), 7.09-7.14 (m, 2H), 7.27 (td, J=8.9, 2.6 Hz, 1H), 7.46 (dd, J=10.5, 2.6 Hz, 1H), 7.87 (d, J=8.6 Hz, 1H), 8.12 (dd, J=9.2, 5.9 Hz, 1H).

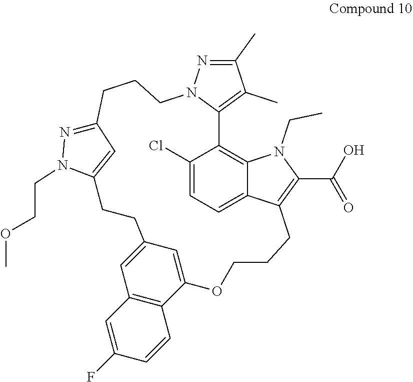 [SMILES: CCn1c(C(=O)O)c2c3ccc(Cl)c(c31)-c1c(C)c(C)nn1CCCc1cc(n(CCOC)n1)CCc1cc(c3ccc(F)cc3c1)OCCC2]

Sₐ or Rₐ; one atropisomer but absolute stereochemistry undetermined

A solution of LiOH (23.7 mg, 0.991 mmol, 15 eq.) in water (0.5 mL) was added to a solution of Intermediate 63 (48 mg, 0.0661 mmol) in THE (1 mL) and MeOH (1 mL). The reaction was stirred at 60° C. for 3 h. After cooling, the reaction mixture was diluted with MeOH and this solution was directly injected into preparative HPLC (Stationary phase: RP XBridge Prep C18 OBD-10 μm, 30×150 mm, Mobile phase: 0.25% NH₄HCO₃ solution in water, CH₃CN). The resulting solid was suspended in DIPE and evaporated to give Compound 10 (43 mg, yield: 91%) as a yellow powder.

¹H NMR (400 MHz, DMSO-d₆) δ ppm 0.84 (t, J=6.9 Hz, 3H), 1.40-1.52 (m, 1H), 1.55-1.67 (m, 1H), 1.77 (s, 3H), 2.11 (t, J=7.4 Hz, 2H), 2.16 (s, 3H), 2.18-2.26 (m, 2H), 2.84-2.96 (m, 2H), 2.96-3.11 (m, 3H), 3.12 (s, 3H), 3.15-3.25 (m, 1H), 3.42-3.54 (m, 5H), 3.78-3.97 (m, 4H), 4.37-4.48 (m, 1H), 5.10 (s, 1H), 6.48 (s, 1H), 7.09 (d, J=8.5 Hz, 1H), 7.13 (s, 1H), 7.25 (td, J=8.9, 2.6 Hz, 1H), 7.45 (dd, J=10.5, 2.6 Hz, 1H), 7.80 (d, J=8.6 Hz, 1H), 8.10 (dd, J=9.2, 5.9 Hz, 1H).

### Analytical Analysis

### LCMS Methods

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica.

LCMS Method Codes (Flow expressed in mL/min; column temperature (T) in ° C.; Run time in minutes)

| Method | | | | | Flow | Run |
|---|---|---|---|---|---|---|
| Code | Instrument | column | mobile phase | gradient | Col T | time |
| 1 | Waters: Acquity ® | Waters: BEH | A: 10 mM CH₃COONH₄ in | From 100% A to | 0.7 | 3.5 |
| | UPLC ® - | (1.8 μm, | 95% H₂O + 5% CH₃CN | 5% A in 2.10 min, to | 55 | |
| | DAD and SQD | 2.1*100 mm) | B: CH₃CN | 0% A in 0.90 min, to | | |
| | | | | 5% A in 0.5 min | | |
| 2 | Waters: Acquity ® | Waters: BEH | A: 0.1% NH₄HCO₃ in | From 100% A to | 0.6 | 4.5 |
| | UPLC ® - | (1.8 μm, | 95% H₂O + 5% CH₃CN | 5% A in 2.10 min, to | 55 | |
| | DAD and SQD | 2.1 × 100 mm) | B: CH₃CN | 0% A in 0.9 min, to | | |
| | | | | 5% A in 0.5 min | | |
| 3 | Waters: Acquity ® | Waters: BEH | A: 10 mM CH₃COONH₄ in | From 100% A to | 0.6 | 3.5 |
| | UPLC ® - | (1.8 μm, | 95% H₂O + 5% CH₃CN | 5% A in 2.10 min, to | 55 | |
| | DAD and SQD | 2.1 × 100 mm) | B: CH₃CN | 0% A in 0.9 min, to | | |
| | | | | 5% A in 0.5 min | | |

| Compound | |
|---|---|
| number | LCMS results |
| 1 | confirms the MW (RT: 1.84, [M + H]+ 654, Method: 1) |
| 2 | confirms the MW (RT: 1.84, [M + H]+ 654, Method: 1) |
| 3 | confirms the MW (RT: 1.86, [M + H]⁺ 668, Method: 2) |
| 4 | confirms the MW (RT: 1.86, [M + H]⁺ 668, Method: 2) |
| 5 | confirms the MW (RT: 1.93, [M + H]⁺ 742, Method: 3) |
| 6 | confirms the MW (RT: 1.93, [M + H]⁺ 742, Method: 3) |
| 7 | confirms the MW (RT: 2.05, [M + H]⁺ 725, Method: 3) |
| 8 | confirms the MW (RT: 2.05, [M + H]⁺ 725, Method: 3) |
| 9 | confirms the MW (RT: 1.92, [M + H]⁺ 712, Method: 3) |
| 10 | confirms the MW (RT: 1.92, [M + H]⁺ 712, Method: 3) |

### SFC Methods:

The SFC measurement was performed using an Analytical Supercritical fluid chromatography (SFC) system composed by a binary pump for delivering carbon dioxide (CO2) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software. Analytical SFC-MS Methods (Flow expressed in mL/min; column temperature (Col T) in CC; Run time in minutes, Backpressure (BPR) in bars. “iPrNH₂” means isopropylamine, “iPrOH” means 2-propanol, “EtOH” means ethanol, “min” mean minutes.

| Method | | | | Flow | Run time |
|---|---|---|---|---|---|
| code | column | mobile phase | gradient | Col T | BPR |
| 1 | Daicel | A: CO₂ | 10%-50% | 2.5 | 9.5 |
| | Chiralpak ® IG3 | B: EtOH + | B in 6 min, | 40 | 130 |
| | column (3.0 μm, | 0.2% iPrNH₂ | hold 3.5 min | | |
| | 150 × 4.6 mm) | | | | |
| 2 | Daicel | A: CO₂ | 10%-50% | 2.5 | 9.5 |
| | Chiralpak ® IH3 | B: EtOH + | B in 6 min, | 40 | 130 |
| | column (3.0 μm, | 0.2% iPrNH₂ | hold 3.5 min | | |
| | 150 × 4.6 mm) | | | | |

TABLE
Analytical SFC data - Rₜ means retention time (in minutes),
[M + H]⁺ means the protonated mass of the compound,
method refers to the method used for (SFC)MS
analysis of enantiomerically pure compounds. No. means number.

| Co. No. | SFC Method | Rt | [M + H]⁺ |
|---|---|---|---|
| 3 | 1 | 6.02 | 668 |
| 4 | 1 | 6.46 | 668 |
| 5 | 2 | 4.18 | 742 |
| 6 | 2 | 3.78 | 742 |
| 7 | 1 | 5.91 | 725 |
| 8 | 1 | 7.38 | 725 |
| 9 | 2 | 3.62 | 712 |
| 10 | 2 | 3.21 | 712 |

### NMR

¹H NMR spectra were recorded on Bruker Avance III 400 MHz and Avance NEO 400 MHz spectrometers. CDCl₃ was used as solvent, unless otherwise mentioned. The chemical shifts are expressed in ppm relative to tetramethylsilane.

### Pharmacological Analysis

### Biological Example 1

Terbium labeled Myeloid Cell Leukemia 1 (Mcl-1) homogeneous time-resolved fluorescence (HTRF) binding assay utilizing the BIM BH3 peptide (H2N-(C/Cy5Mal) WIAQELRRIGDEFN-OH) as the binding partner for Mcl-1.

Apoptosis, or programmed cell death, ensures normal tissue homeostasis, and its dysregulation can lead to several human pathologies, including cancer. Whilst the extrinsic apoptosis pathway is initiated through the activation of cell-surface receptors, the intrinsic apoptosis pathway occurs at the mitochondrial outer membrane and is governed by the binding interactions between pro- and anti-apoptotic Bcl-2 family proteins, including Mcl-1. In many cancers, the anti-apoptotic Bcl-2 protein(s), such as the Mcl-1, are upregulated, and in this way the cancer cells can evade apoptosis. Thus, inhibition of the Bcl-2 protein(s), such as Mcl-1, may lead to apoptosis in cancer cells, providing a method for the treatment of said cancers.

This assay evaluated inhibition of the BH3 domain: Mcl-1 interaction by measuring the displacement of Cy5-labeled BIM BH3 peptide (H₂N—(C/Cy5Mal) WIAQELRRIGDEFN-OH) in the HTRF assay format.

### Assay Procedure

The following assay and stock buffers were prepared for use in the assay: (a) Stock buffer: 10 mM Tris-HCl, pH=7.5+150 mM NaCl, filtered, sterilized, and stored at 4° C.; and (b) 1×assay buffer, where the following ingredients were added fresh to stock buffer: 2 mM dithiothreitol (DTT), 0.0025% Tween-20, 0.1 mg/mL bovine serum albumin (BSA). The 1×Tb-Mcl-1+Cy5 Bim peptide solution was prepared by diluting the protein stock solution using the 1×assay buffer (b) to 25 pM Tb-Mcl-1 and 8 nM Cy5 Bim peptide.

Using the Acoustic ECHO, 100 nL of 100×test compound(s) were dispensed into individual wells of a white 384-well Perkin Elmer Proxiplate, for a final compound concentration of 1× and final DMSO concentration of 1%. Inhibitor control and neutral control (NC, 100 nL of 100% DMSO) were stamped into columns 23 and 24 of assay plate, respectively. Into each well of the plate was then dispensed 10 μL of the 1×Tb-Mcl-1+Cy5 Bim peptide solution. The plate was centrifuged with a cover plate at 1000 rpm for 1 minute, then incubated for 60 minutes at room temperature with plates covered. The TR-FRET signal was read on an BMG PHERAStar FSX MicroPlate Reader at room temperature using the HTRF optic module (HTRF: excitation: 337 nm, light source: laser, emission A: 665 nm, emission B: 620 nm, integration start: 60 μs, integration time: 400 μs).

### Data Analysis

The BMG PHERAStar FSX MicroPlate Reader was used to measure fluorescence intensity at two emission wavelengths—665 nm and 620 nm—and report relative fluorescence units (RFU) for both emissions, as well as a ratio of the emissions (665 nm/620 nm)*10,000. The RFU values were normalized to percent inhibition as follows:

% inhibition=(((NC−IC)−(compound−IC))/(NC−IC))*100

where IC (inhibitor control, low signal)=mean signal of 1×Tb-MCl-1+Cy5 Bim peptide+inhibitor control or 100% inhibition of Mcl-1; NC (neutral control, high signal)=mean signal 1×Tb-MCl-1+Cy5 Bim peptide with DMSO only or 0% inhibition

An 11-point dose response curve was generated to determine IC₅₀ values (using GenData) based on the following equation:

Y=Bottom+(Top−Bottom)/(1+10\hat{ }((log IC₅₀−X)*HillSlope))

where Y=% inhibition in the presence of X inhibitor concentration; Top=100% inhibition derived from the IC (mean signal of Mcl-1+inhibitor control); Bottom=0% inhibition derived from the NC (mean signal of Mcl-1+DMSO); Hillslope=Hill coefficient; and IC₅₀=concentration of compound with 50% inhibition in relation to top/neutral control (NC).

Ki=IC₅₀/(1+[L]/Kd)

In this assay [L]=8 nM and Kd=10 nM

Representative compounds of the present invention were tested according to the procedure as described above, with results as listed in the Table below (n.d. means not determined).

| Compound | Tb-MCLl Ki (nM) |
|---|---|
| 1 | 0.090 |
| 2 | 3.21 |
| 3 | 0.043 |
| 4 | 33.7 |
| 5 | 0.027 |
| 6 | 1.93 |
| 7 | 0.052 |
| 8 | 8.06 |
| 9 | 0.061 |
| 10 | 3.06 |

### Biological Example 2

MCL-1 is a regulator of apoptosis and is highly over-expressed in tumor cells that escape cell death. The assay evaluates the cellular potency of small-molecule compounds targeting regulators of the apoptosis pathway, primarily MCL-1, Bfl-1, Bcl-2, and other proteins of the Bcl-2 family. Protein-protein inhibitors disrupting the interaction of anti-apoptotic regulators with BH3-domain proteins initiate apoptosis.

The Caspase-Glo® 3/7 Assay is a luminescent assay that measures caspase-3 and -7 activities in purified enzyme preparations or cultures of adherent or suspension cells. The assay provides a proluminescent caspase-3/7 substrate, which contains the tetrapeptide sequence DEVD. This substrate is cleaved to release aminoluciferin, a substrate of luciferase used in the production of light. Addition of the single Caspase-Glo® 3/7 Reagent in an “add-mix-measure” format results in cell lysis, followed by caspase cleavage of the substrate and generation of a “glow-type” luminescent signal.

This assay uses the MOLP-8 human multiple myeloma cell line, which is sensitive to MCL-1 inhibition.

### Materials:

Perkin Elmer Envision
Multidrop 384 and small volume dispensing cassettes
Centrifuge
Countess automated cell counter
Countess counting chamber slides
Assay plate: ProxiPlate-384 Plus, White 384-shallow well Microplate
Sealing tape: Topseal A plus
T175 culture flask

| Product | Units | | Storage |
|---|---|---|---|
| RPMI1640 (no L-Glutamine, no | 500 | mL | 4° C. |
| phenol red) | | | |
| Foetal Bovine Serum (FBS) (Heat | 500 | mL | 4° C. |
| inactivated) | | | |
| L-Glutamine (200 mM) | 100 | ml | −20° C. |
| Gentamicin (50 mg/mL) | 100 | mL | 4° C. |
| Caspase 3/7 Detection kit | 100 | mL | −20° C. |
| | 10 × 10 | mL | |

### Cell Culture Media:

| MOLP8 | | |
|---|---|---|
| RPMI-1640 medium | 500 | mL |
| 20% FBS (heat inactivated) | 120 | mL |
| 2 mM L-Glutamine | 6.2 | mL |
| 50 μg/mL Gentamicin | 620 | μL |
| Assay media | | |
| RPMI-1640 medium | 500 | mL |
| 10% FBS (Heat inactivated) | 57 | mL |
| 2 mM L-Glutamine | 5.7 | mL |
| 50 μg/mL Gentamicin | 570 | μL |

### Cell Culture:

Cell cultures were maintained between 0.2 and 2.0×10⁶ cells/mL. The cells were harvested by collection in 50 mL conical tubes. The cells were then pelleted at 500 g for 5 mins before removing supernatant and resuspension in fresh pre-warmed culture medium. The cells were counted and diluted as needed.

### Caspase-Glo Reagent:

The assay reagent was prepared by transferring the buffer solution to the substrate vial and mixing. The solution may be stored for up to 1 week at 4° C. with negligible loss of signal.

### Assay Procedure:

Compounds were delivered in assay-ready plates (Proxiplate) and stored at −20° C. Assays always include 1 reference compound plate containing reference compounds. The plates were spotted with 40 nL of compounds (0.5% DMSO final in cells; serial dilution; 30 μM highest conc. 1/3 dilution, 10 doses, duplicates). The compounds were used at room temperature and 4 μL of pre-warmed media was added to all wells except column 2 and 23. The negative control was prepared by adding 1% DMSO in media. The positive control was prepared by adding the appropriate positive control compound in final concentration of 60 μM in media. The plate was prepared by adding 4 μL negative control to column 23, 4 μL positive control to column 2 and 4 μL cell suspension to all wells in the plate. The plate with cells was then incubated at 37° C. for 2 hours. The assay signal reagent is the Caspase-Glo solution described above, and 8 μL was added to all wells. The plates were then sealed and measured after 30 minutes.

The activity of a test compound was calculated as percent change in apoptosis induction as follows:

$$\begin{matrix}LC = median of the Low Control values \\ = Central Reference in Screener \\ = DMSO \\ = 0 \% \\ HC = Median of the High Control values \\ = Scale Reference in Screener \\ = 30 μ M of positive control \\ = 100 \% apoptosis induction\end{matrix}$$

% Effect (AC₅₀)=100−((sample−LC)/(HC−LC))*100

% Control=(sample/HC)*100

% Control min=(sample−LC)/(HC−LC)*100

TABLE
Measured AC₅₀ for Representative Compounds of Formula (I). Averaged
values are reported over all runs on all batches of a particular compound.

| | MOLP8 Caspase- |
|---|---|
| Compound | Glo AC₅₀ (μM) |
| 1 | 0.067 |
| 2 | 2.71 |
| 3 | 0.036 |
| 4 | 13.13 |
| 5 | 0.027 |
| 6 | 1.93 |
| 7 | 0.016 |
| 8 | 1.56 |
| 9 | 0.053 |
| 10 | 3.06 |

NT = not tested

1. A compound of Formula (I): 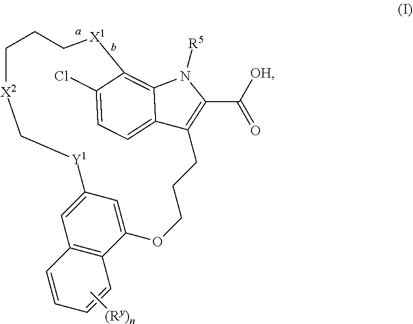
or a tautomer or a stereoisomeric form thereof, wherein:
X¹ is 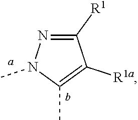
wherein ‘a’ and ‘b’ indicate how variable X¹ is attached to the remainder of the molecule;
R^{y} is halo;
n is 0, 1 or 2;
X² is 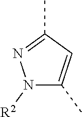
which can be attached to the remainder of the molecule in both directions;
R¹ represents is hydrogen; or C₁₋₆alkyl optionally substituted with one substituent that is Het¹, —OR³, or —NR^{4a}R^{4b};
R² is hydrogen; methyl; or C₂₋₆alkyl optionally substituted with one substituent that is Het¹, —OR³, or —NR^{4a}R^{4b};
R^{1a} is methyl or ethyl;
R³ represents is hydrogen, C₁₋₄alkyl, or —C₂₋₄alkyl-O—C₁₋₄alkyl;
R^{4a} and R^{4b} are each, independently, hydrogen or C₁₋₄alkyl;
R⁵ is hydrogen; methyl; or C₂₋₆alkyl optionally substituted with one substituent that is C₃₋₆cycloalkyl, Het¹, —NR^{4a}R^{4b}, or —OR³;
Het¹ is a 4- to 7-membered monocyclic fully saturated heterocyclyl containing one or two heteroatoms each, independently, O, S, or N, wherein said S-atom is optionally substituted to form S(═O) or S(═O)₂; wherein said heterocyclyl is optionally substituted with one or two substituents that are each, independently, halo, cyano, or —O—C₁₋₄alkyl;
Y¹ is —(CH₂)ₘ— or —S—;
m is 1 or 2;
or a pharmaceutically acceptable salt, or a solvate thereof.

2. The compound according to claim 1, wherein:
n is 0 or 1;
R¹ is methyl;
R² is methyl;
R^{1a} is methyl;
R⁵ is methyl; or C₂₋₆alkyl optionally substituted with one —OR³;
R³ is —C₂₋₄alkyl-O—C₁₋₄alkyl;
Y¹ is —S—.

3. The compound according to claim 2, wherein n is 0.

4. The compound according to claim 2, wherein n is 1.

5. The compound according to claim 1, wherein Y¹ is —S—.

6. The compound according to claim 1, wherein R⁵ is C₂₋₆alkyl optionally substituted with one —OR³.

7. The compound according to claim 4, wherein R^{y} is fluoro.

8. A pharmaceutical composition comprising the compound of claim 1 and a pharmaceutically acceptable carrier or diluent.

9. A process for preparing the pharmaceutical composition of claim 8 comprising mixing a pharmaceutically acceptable carrier with a therapeutically effective amount of the compound of claim 1.

10-11. (canceled)

12. The method of claim 13, wherein the cancer is prostate cancer, lung cancer, pancreatic cancer, breast cancer, ovarian cancer, cervical cancer, melanoma, B-cell chronic lymphocytic leukemia (CLL), acute myeloid leukemia (AML), or acute lymphoblastic leukemia (ALL).

13. A method of treating or preventing cancer, comprising administering to a subject in need thereof, a therapeutically effective amount of the compound of claim 1.

14. A method of treating or preventing cancer, comprising administering to a subject in need thereof, the compound pharmaceutical composition of claim 8.

15. The method of claim 15, wherein the cancer is prostate cancer, lung cancer, pancreatic cancer, breast cancer, ovarian cancer, cervical cancer, melanoma, B-cell chronic lymphocytic leukemia (CLL), acute myeloid leukemia (AML), or acute lymphoblastic leukemia (ALL).
